(19) United States
(12) Patent Application Publication
(10) Pub. No.: US 2024/0189327 A1
(43) Pub. Date: Jun. 13, 2024

(54) SYNERGISTIC CANNABINOID ESTERS, THEIR SALTS AND USES THEREOF

(71) Applicant: LONDON PHARMACEUTICALS AND RESEARCH CORPORATION, London (CA)

(72) Inventor: Mahmoud Mohamed Abdrabo Moustafa, London, Ontario (CA)

(21) Appl. No.: 18/279,974

(22) PCT Filed: Mar. 3, 2022
(86) PCT No.: PCT/CA2022/050304

§ 371 Date: Sep. 1, 2023

Related U.S. Application Data
(60) Provisional application No. 63/155,949, filed on Mar. 3, 2021.

(51) Int. Cl. A61K 31/00; A61K 9/06; A61K 9/20; A61K 9/48; A61K 9/50; A61K 31/7032; A61K 47/10; A61K 47/14; A61K 47/26

(52) U.S. Cl. CPC A61K 31/658; A61K 9/06; A61K 9/2009; A61K 9/2018; A61K 9/2027; A61K 9/2054; A61K 9/2059; A61K 9/4858; A61K 9/4866; A61K 9/50; A61K 31/7032; A61K 47/10; A61K 47/14; A61K 47/26

(57) ABSTRACT

Cannabinoid esters and their soluble salts with synergistic or additive therapeutic counterparts and stable formulations thereof, as well as their edible, beverage and medicinal applications. The synergistic or additive cannabinoid esters may be used as drugs or prodrugs for treating various conditions related to the modulation or biased modulation of cannabinoid receptors, including but not limited to, pain and inflammation, arthritis, cancer, glaucoma, neurodegenerative disorders, multiple sclerosis, renal fibrosis, fibrotic disorder, mental health disorders, addiction, motor function disorders and gastrointestinal and metabolic disorders.

### FIELD OF THE INVENTION

The present invention relates to the field of medicinal chemistry and, in particular, to cannabinoid esters, their salts with synergistic or additive therapeutic counterparts and uses for treating, alleviating, or reducing symptoms of illnesses in human or animal subjects.

### BACKGROUND

The endocannabinoid system mediates many important physiological functions including neuroplasticity and learning, emotion and motivation, appetite, and GI motility as well as immunomodulation. There are at least two types of G-protein coupled cannabinoid receptors that have been isolated and fully characterized in mammals: a) CB1: located centrally and peripherally and involved mainly in neurotransmitters homeostasis; and b) CB2: located peripherally and linked with the immune system. These receptors represent a promising therapeutic target for various conditions including chronic pain, inflammation, neurodegenerative disorders, epilepsy, addiction, insomnia, cancer, obesity, and anorexia. Designing specific cannabinoid ligands to manage these conditions has received increased interest in recent years.

The cannabinoid receptors can be modulated by a heteromorphic group of compounds, so-called cannabinoids. They can be classified into three main groups: a) endogenous or endocannabinoids (e.g. arachidonoylethanolamide); b) natural or phytocannabinoids, which are the active constituents of Cannabis species (e.g. delta-9-tetrahydrocannabinol (THC) and cannabidiol (CBD)); and c) synthetic (e.g. nabilone), which are illustrated in Table 1, below.

TABLE 1
Representative examples of cannabinoids

| Cannabinoids | |
|---|---|
| class | Examples |
| A. Endogenous | 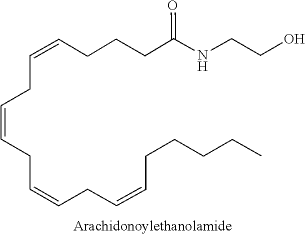 [SMILES: CCCCC/C=C\C/C=C\C/C=C\C/C=C\CCCC(=O)NCCO] |
| B. Natural | 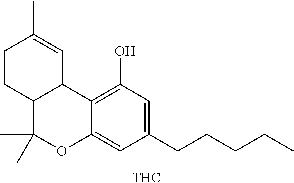 |
| | 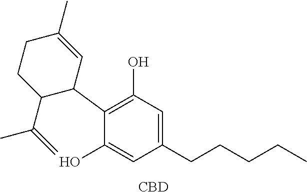 |
| C. Synthetic | 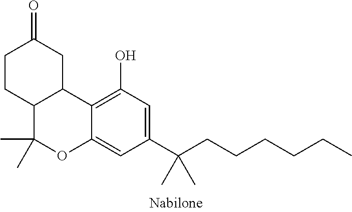 [SMILES: CCCCCCC(C)(C)c1cc(O)c2c(c1)OC(C)(C)C1CCC(=O)CC21] |

The clinical utility of cannabinoids has been documented in the treatment of many conditions. Sativex®, by GW Pharmaceuticals, is a buccal spray of THC and CBD in a 1:1 mixture and has been approved in many countries as an adjunctive treatment of neuropathic pain and spasticity associated with multiple sclerosis in adults. Cesamet™ (nabilone), by Bausch Health Co., is a synthetic cannabinoid for oral administration as an antiemetic through a CB1 receptor mediated interaction.

Despite their clinical potential, natural cannabinoids (phytocannabinoids) extracted from C. Sativa are highly lipophilic (possessing log P values of 6-7), sparingly soluble in water (aqueous solubility=2-10 μg/mL at 23° C.), chemically unstable (particularly in solution via light, temperature, and auto-oxidation), and gummy in nature with erratic absorption, a delayed onset, extensive first-pass metabolism, high plasma protein binding, large volume of distribution and low systemic bioavailability after oral administration, leading to unpredictable time course of action and long half-life (t_{1/2}). In addition, the clinical benefits of smoked herb are short and associated with mucosal damage, serious adverse effects, and exposure to carcinogenic by-products. Furthermore, THC can cross the blood brain barrier (BBB) and activate central CB1 producing unwanted psychotropic effects. In an attempt to overcome these limitations, a variety of formulations and drug delivery approaches have been developed including co-solvency, complexation, surfactant and carrier-assisted methods, thus far, with limited success.

On the other hand, several synthetic derivatives and pro-drugs have been reported and widely used to modulate CB1 and CB2. For example, WO 2017/216362 A1 of Full Spectrum Laboratories Ltd. discloses cannabinoid prodrugs, and their production, formulations and uses. This reference discloses only esters of organic and amino acids, for example, succinic acid and valine.

WO 2004/043946 A1 of Mallinckrodt Inc. discloses highly crystalline aryl sulfonic THC esters. They can be recrystallized for purification and are stable at room temperature in air, allowing for indefinite storage and recovery of pure THC upon hydrolysis. This reference does not disclose any pharmacological actions or clinical utilities for these esters.

Watanabe et al (Chem Pharm Bull 27: 3009-3014, 1979) reported the chemical synthesis of delta-8-THC glucuronide and sulfate esters to study their hydrolysis, acute toxicity and metabolic disposition in rats. However, no pharmacological or biological utility is described for any of theses esters.

Juntunen et al (Eur. J. Pharm. Sci. 19, 37-43, 2003) reported the synthesis of a water-soluble phosphate ester pro-drug of anandamide. The phosphate functional group increased the aqueous solubility of the parent endocannabinoids by >16 500-fold at pH 7.4 and reduced the intraocular pressure in normotensive rabbit. The study did not investigate any of the more clinically useful phytocannabinoids and did not report any other pharmacological actions or clinical uses of this phosphate ester of anandamide or its salts.

On the other hand, there are known clinical benefits of cannabinoids in combinations with other therapeutic agents either by separate, sequential, or simultaneous administration. For example, WO2020021545A1 of BOL Pharma Ltd., Israel; WO2020009950A1 of Companion Sciences, LLC, USA; U.S. Pat. No. 10,398,776B1 of Essential Green Goodness LLC, USA disclose a combination therapy comprising individual therapeutic agents including cannabidiol (CBD), chondroitin, glucosamine, and methylsulfonylmethane (MSM), for treating inflammatory joint disease or reducing pain. Although promising, this therapeutic approach is hampered by the fact that these drugs exhibit different pharmacokinetic profiles, so they do not reach their target tissues at the same time. In particular, glucosamine reaches its maximum plasma level after 50 mins, while it takes 4-6 hours for CBD to reach its maximum plasma level. The same remarkable difference was also observed in other PK parameters such as distribution, rate of metabolism and elimination. Having two synergistically acting or additive molecules with two different PK behaviors is a known major challenge in drug discovery and development.

To minimize the limitations in the prior art, there exists a demand for new synergistic cannabinoid derivatives with optimized physicochemical, pharmacokinetic (PK) and pharmacodynamic (PD) properties for specific clinical applications.

### SUMMARY OF THE INVENTION

The cannabinoid compounds, according to the present invention, are labile esters of cannabinoids and their salts with other synergistic or additive therapeutic counterparts. These new compounds aim to deliver multiple therapeutic benefits via more than one mechanism of action. This is achieved by having a cannabinoid ester and another counter part with a different therapeutic effect. The esters are sensitive to enzymatic or chemical hydrolysis within the human or animal body, to release the parent cannabinoids and the synergistic or additive therapeutic counterparts thereby modulating the endocannabinoid system and other cooperative receptors and tissues.

In one embodiment, the cannabinoid compounds are sulfate or hemisulfate esters of a cannabinoid and their salts with other synergistic or additive therapeutic counterparts.

In another embodiment, the cannabinoid compounds are mono-, di- or tri-phosphate esters of a cannabinoid and their salts with other synergistic or additive therapeutic counterparts.

In another embodiment, the cannabinoid compounds are carbonate esters of a cannabinoid with other synergistic or additive therapeutic counterparts.

In another embodiment, the cannabinoid compounds are carbamate esters of a cannabinoid with other synergistic or additive therapeutic counterparts.

In another embodiment, the cannabinoid compounds are nitrate esters of a cannabinoid and other synergistic or additive therapeutic counterparts.

In another embodiment, the cannabinoid compounds are borate esters of a cannabinoid and other synergistic or additive therapeutic counterparts.

In one embodiment, the cannabinoid compounds are sulfonate esters of a cannabinoid and their salts with other synergistic or additive therapeutic counterparts.

In one embodiment, the cannabinoid compounds are phosphonate esters of a cannabinoid and their salts with other synergistic or additive therapeutic counterparts.

In one embodiment, the cannabinoid compounds are bisphosphonate esters of a cannabinoid and their salts with other synergistic or additive therapeutic counterparts.

In another embodiment, the cannabinoid compounds are sulfate esters of THC, represented by the general formula 1:

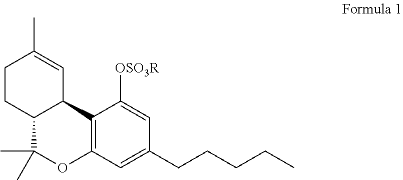

In another embodiment, the cannabinoid compounds are sulfate esters of CBD, represented by the general formula 2 and 3:

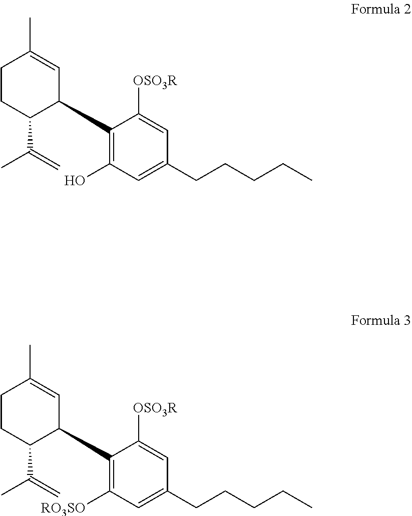

In another embodiment, the cannabinoid compounds are phosphate esters of THC, represented by the general formula 4:

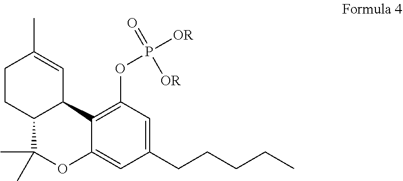

In another embodiment, the cannabinoid compounds are phosphate esters of CBD, represented by the general formula 5 and 6:

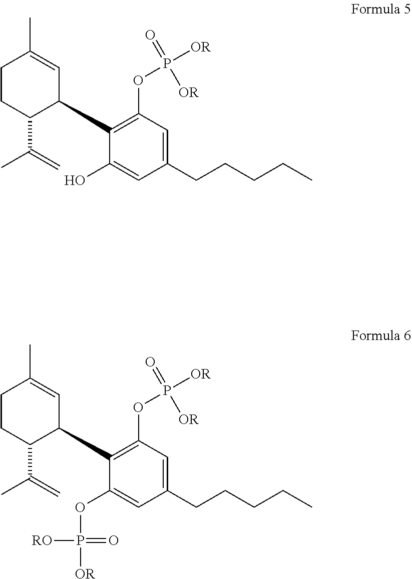

In another embodiment, the cannabinoid compounds are mixed phosphate and sulfate esters of CBD, represented by the general formula 7:

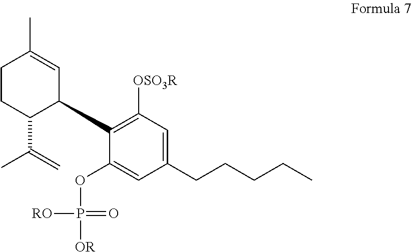

In another embodiment, the cannabinoid compounds are carbonate esters of THC, represented by the general formula 8:

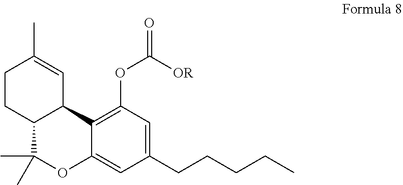

In another embodiment, the cannabinoid compounds are carbonate esters of CBD, represented by the general formula 9 and 10:

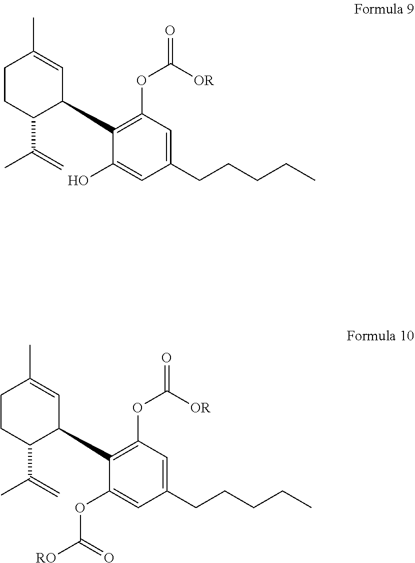

In another embodiment, the cannabinoid compounds are mixed carbonate and sulfate esters of CBD, represented by the general formula 11:

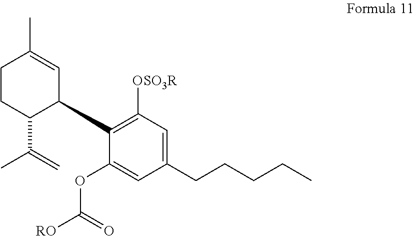

In another embodiment, the cannabinoid compounds are mixed carbonate and phosphate esters of CBD, represented by the general formula 12:

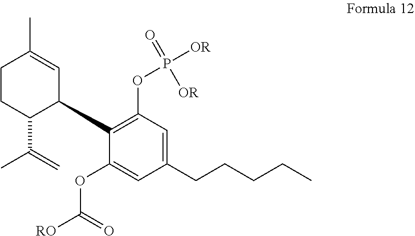

In another embodiment, the cannabinoid compounds are carbamate esters of THC, represented by the general formula 13:

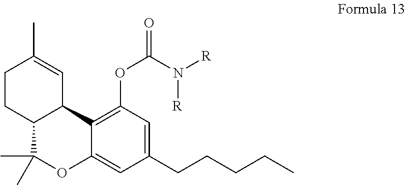

In another embodiment, the cannabinoid compounds are carbamate esters of CBD, represented by the general formula 14 and 15:

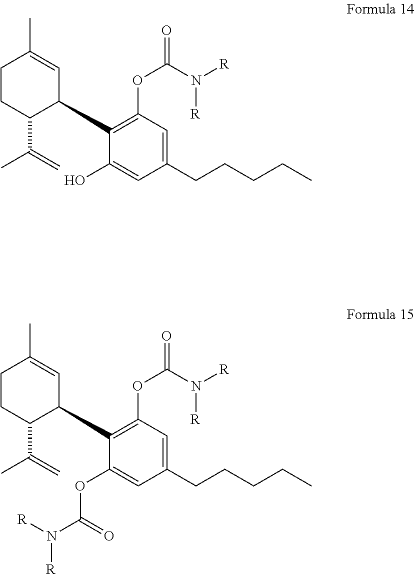

In another embodiment, the cannabinoid compounds are mixed carbamate and sulfate esters of CBD, represented by the general formula 16:

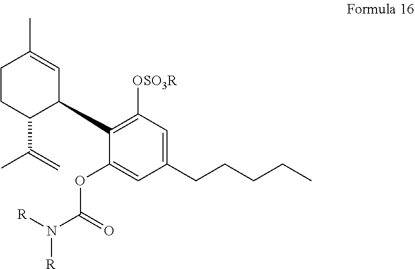

In another embodiment, the cannabinoid compounds are mixed carbamate and phosphate esters of CBD, represented by the general formula 17:

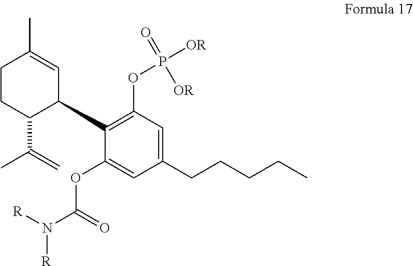

In another embodiment, the cannabinoid compounds are nitrate esters of THC, represented by the general formula 18:

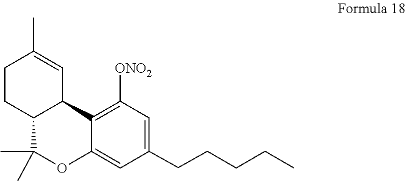

In another embodiment, the cannabinoid compounds are nitrate esters of CBD, represented by the general formula 19 and 20:

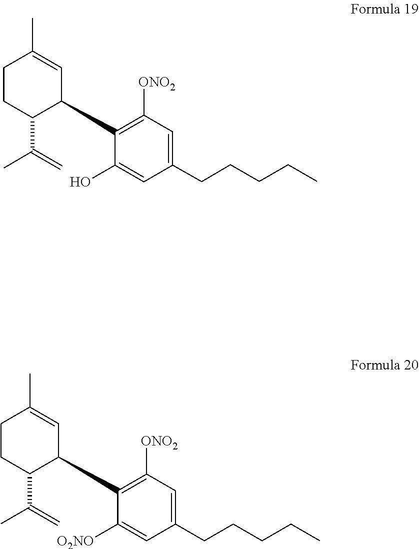

In another embodiment, the cannabinoid compounds are mixed nitrate and sulfate esters of CBD, represented by the general formula 21:

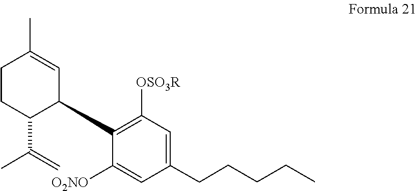

In another embodiment, the cannabinoid compounds are mixed nitrate and phosphate esters of CBD, represented by the general formula 22:

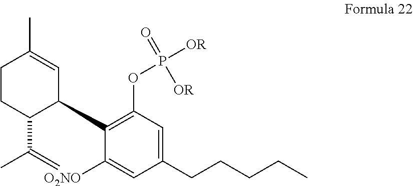

In another embodiment, the cannabinoid compounds are borate esters of THC, represented by the general formula 23:

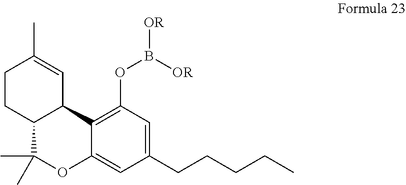

In another embodiment, the cannabinoid compounds are borate esters of CBD, represented by the general formula 24 and 25:

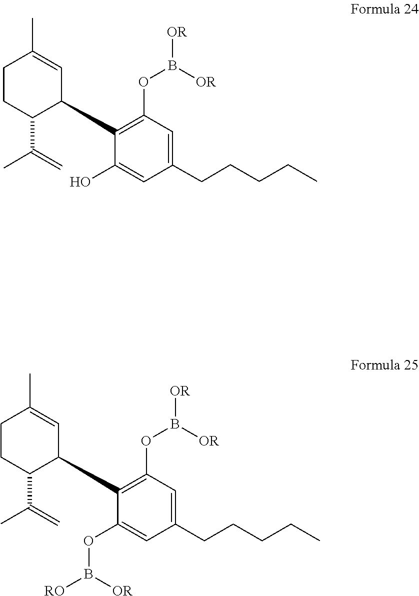

In another embodiment, the cannabinoid compounds are mixed borate and sulfate esters of CBD, represented by the general formula 26:

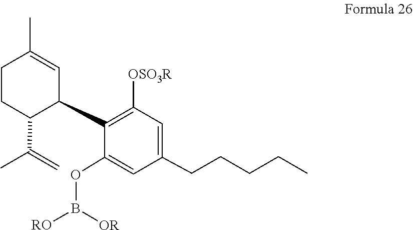

In another embodiment, the cannabinoid compounds are mixed borate and phosphate esters of CBD, represented by the general formula 27:

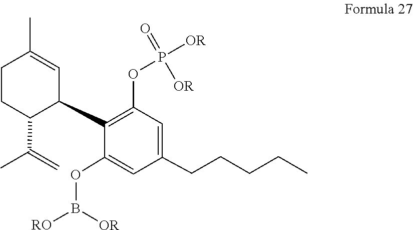

In another embodiment, the cannabinoid compounds are sulfate ester salts of THC, represented by the general formula 28:

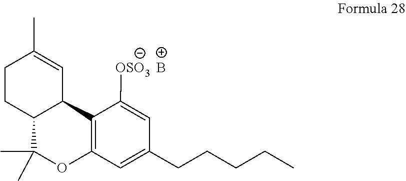

In another embodiment, the cannabinoid compounds are sulfate ester salts of CBD, represented by the general formulas 29 and 30:

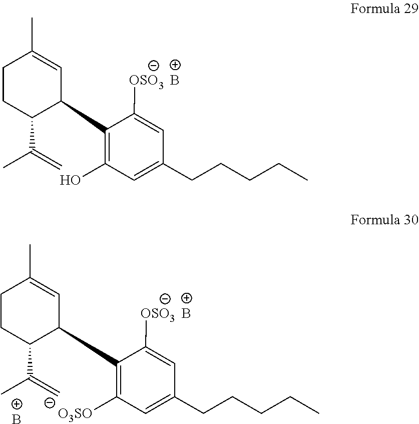

In another embodiment, the cannabinoid compounds are phosphate esters salts of THC, represented by the general formulas 31 and 32:

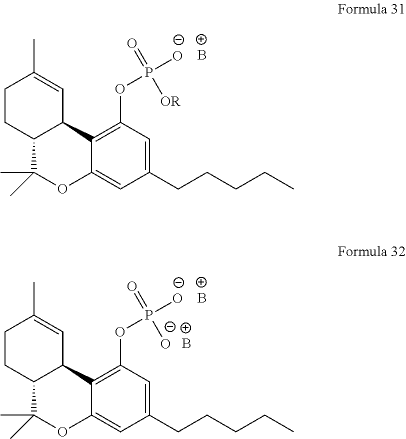

In another embodiment, the cannabinoid compounds are phosphate esters salts of CBD, represented by the general formulas 33-39:

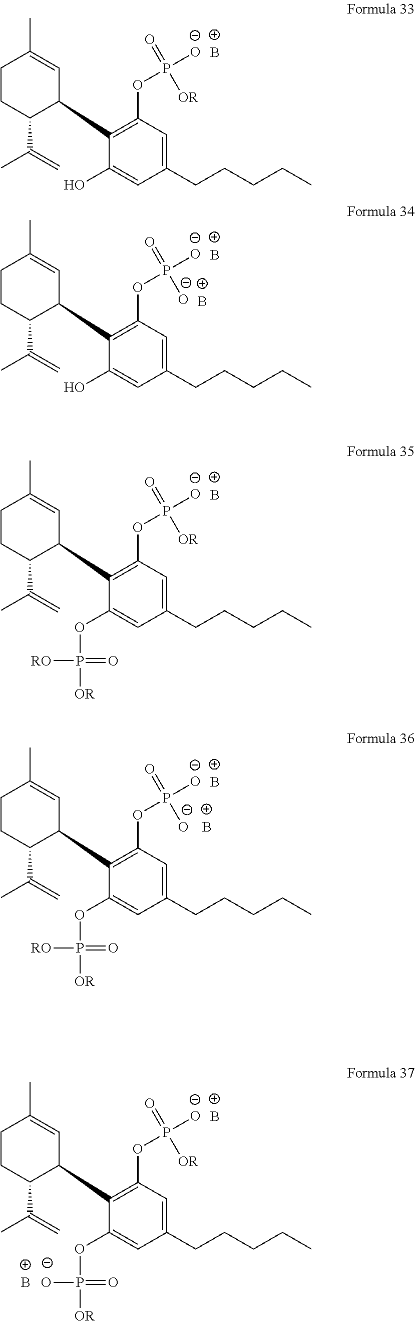

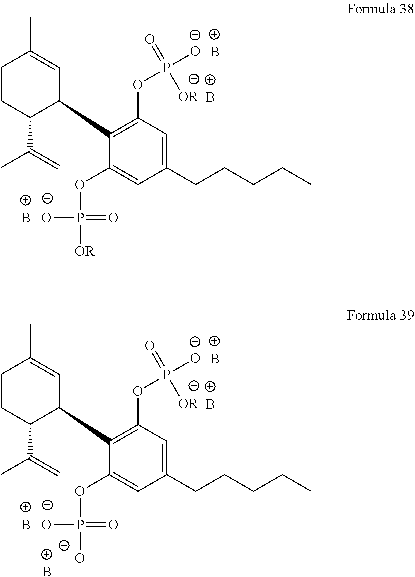

In another embodiment, the cannabinoid compounds are mixed phosphate and sulfate ester salts of CBD, represented by the general formulas 40-43:

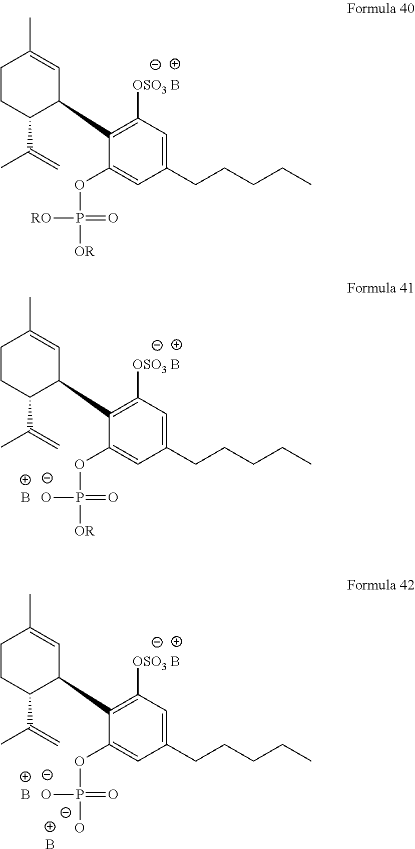

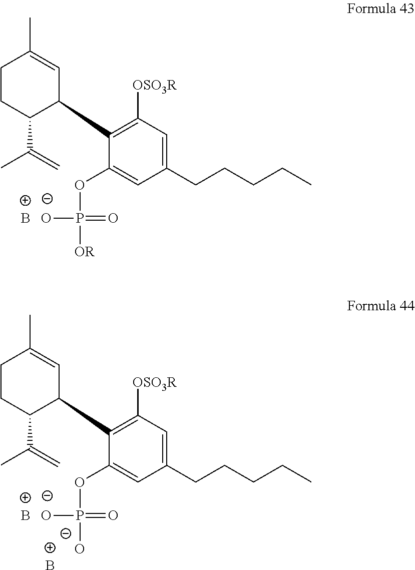

In another embodiment, the cannabinoid compounds are mixed carbonate and sulfate ester salts of CBD, represented by the general formula 45:

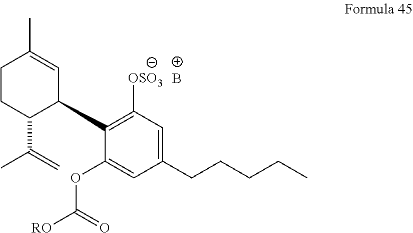

In another embodiment, the cannabinoid compounds are mixed carbonate and phosphate ester salts of CBD, represented by the general formulas 46 and 47:

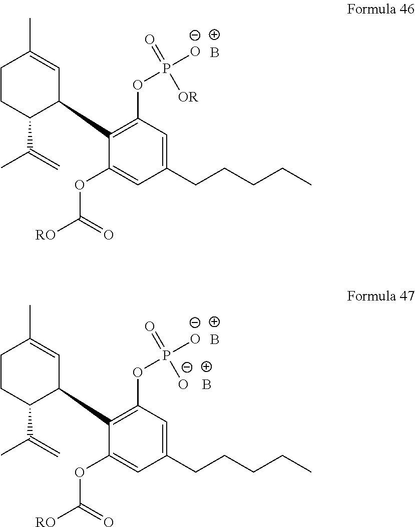

In another embodiment, the cannabinoid compounds are mixed carbamate and sulfate ester salts of CBD, represented by the general formula 48:

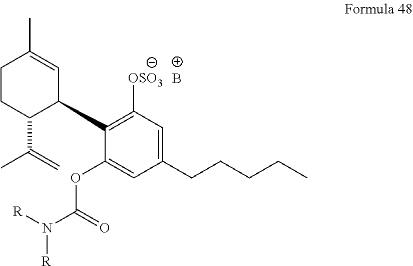

In another embodiment, the cannabinoid compounds are mixed carbamate and phosphate ester salts of CBD, represented by the general formulas 49 and 50:

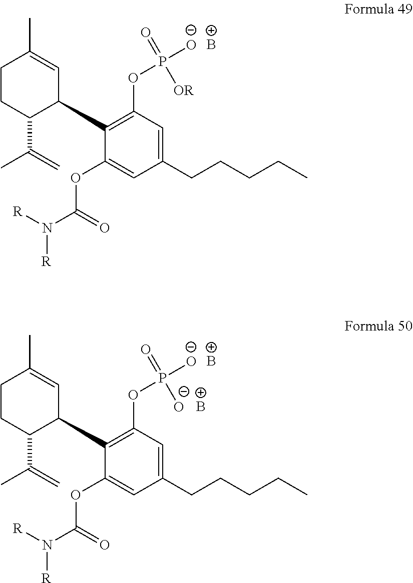

In another embodiment, the cannabinoid compounds are mixed nitrate and sulfate ester salts of CBD, represented by the general formula 51:

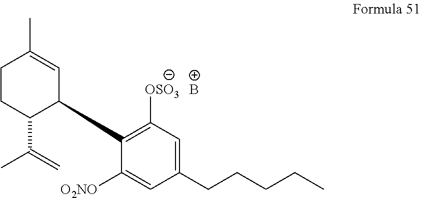

In another embodiment, the cannabinoid compounds are mixed nitrate and phosphate ester salts of CBD, represented by the general formulas 52 and 53:

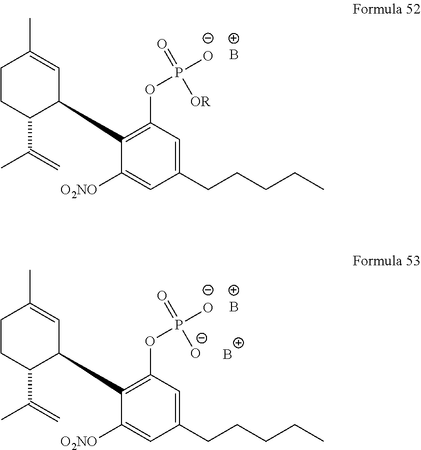

In another embodiment, the cannabinoid compounds are mixed borate and sulfate ester salts of CBD, represented by the general formula 54:

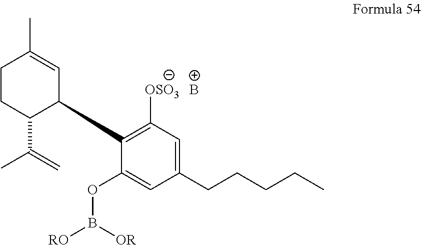

In another embodiment, the cannabinoid compounds are mixed borate and phosphate ester salts of CBD, represented by the general formulas 55 and 56:

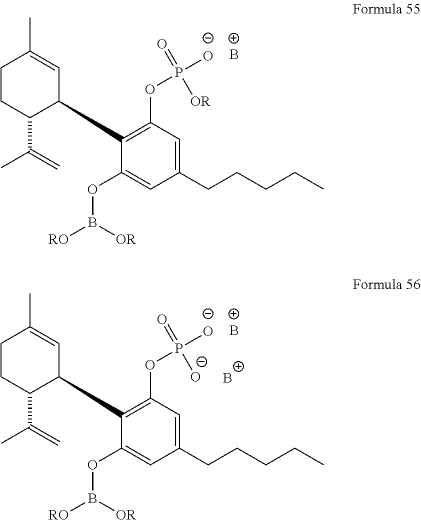

In another embodiment, the cannabinoid compounds are sulfonate esters represented by the general formula 57:

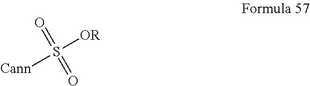

In another embodiment, the cannabinoid compounds are sulfonate esters of THC represented by the general formula 58:

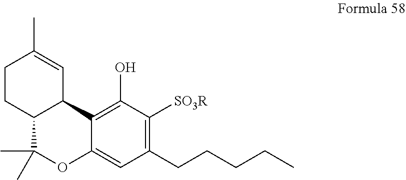

In another embodiment, the cannabinoid compounds are sulfonate esters of CBD represented by the general formula 59:

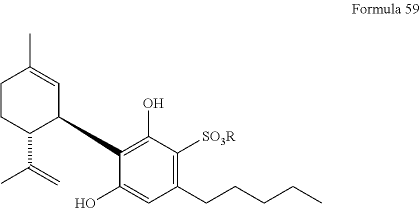

In another embodiment, the cannabinoid compounds are sulfonate ester salts represented by the general formula 60, where “Cann” refers to a cannabinoid and the ester may be linked to any carbon atom on the cannabinoid:

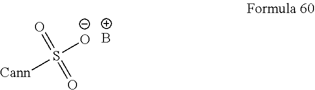

In another embodiment, the cannabinoid compounds are sulfonate ester salts of THC represented by the general formula 61:

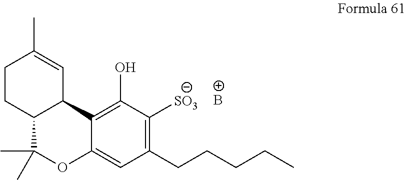

In another embodiment, the cannabinoid compounds are sulfonate ester salts of CBD represented by the general formula 62:

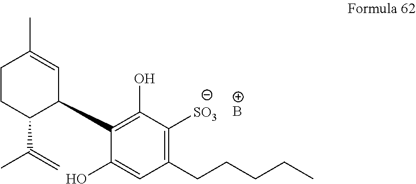

In another embodiment, the cannabinoid compounds are phosphonate esters represented by the general formula 63, where “Cann” refers to a cannabinoid and the ester may be linked to any carbon atom on the cannabinoid:

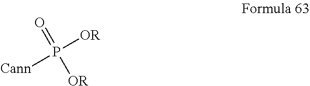

In another embodiment, the cannabinoid compounds are phosphonate esters of THC represented by the general formula 64:

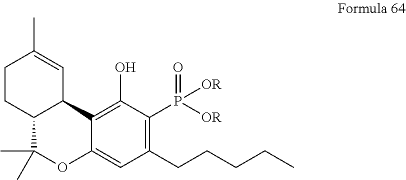

In another embodiment, the cannabinoid compounds are phosphonate esters of CBD represented by the general formula 65:

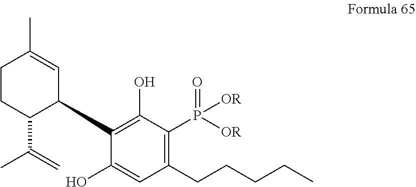

In another embodiment, the cannabinoid compounds are phosphonate ester salts represented by the general formulas 66 and 67, where “Cann” refers to a cannabinoid and the ester may be linked to any carbon atom on the cannabinoid:

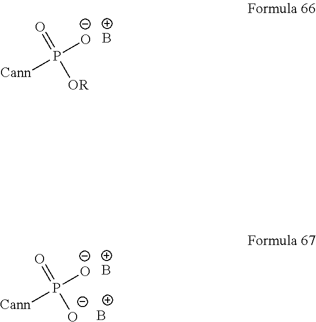

In another embodiment, the cannabinoid compounds are phosphonate ester salts of THC represented by the general formulas 68 and 69:

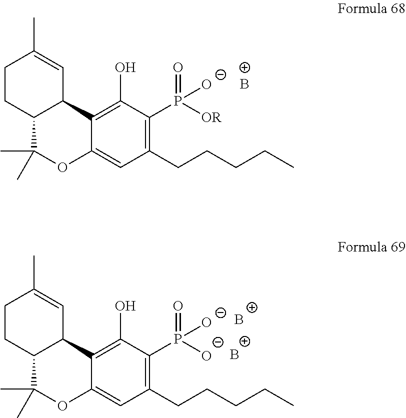

In another embodiment, the cannabinoid compounds are phosphonate ester salts of CBD represented by the general formulas 70 and 71:

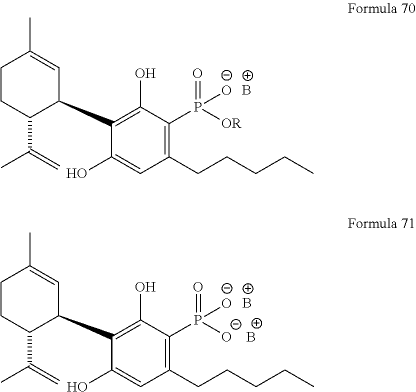

In another embodiment, the cannabinoid compounds are bisphosphonate esters represented by the general formula 72, where “Cann” refers to a cannabinoid and the ester may be linked to any carbon atom on the cannabinoid:

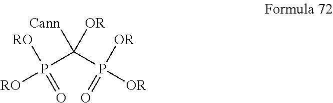

In another embodiment, the cannabinoid compounds are bisphosphonate ester salts represented by the general formulas 73-77, where “Cann” refers to a cannabinoid and the ester may be linked to any carbon atom on the cannabinoid:

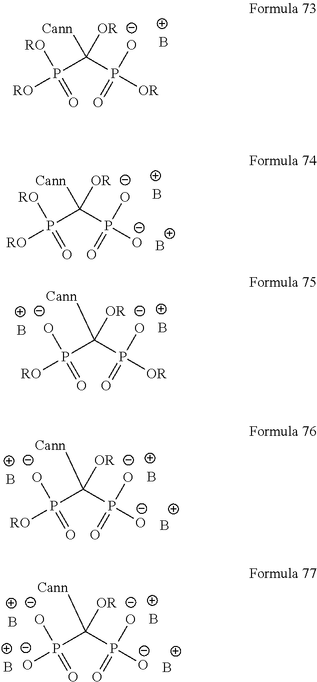

In another embodiment, the cannabinoid compounds are bisphosphonate esters of THC represented by the general formula 78:

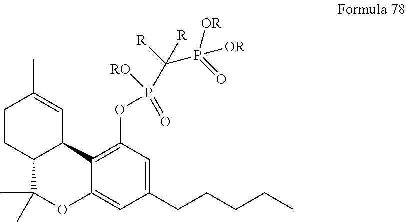

In another embodiment, the cannabinoid compounds are bisphosphonate esters of CBD represented by the general formulas 79 and 80:

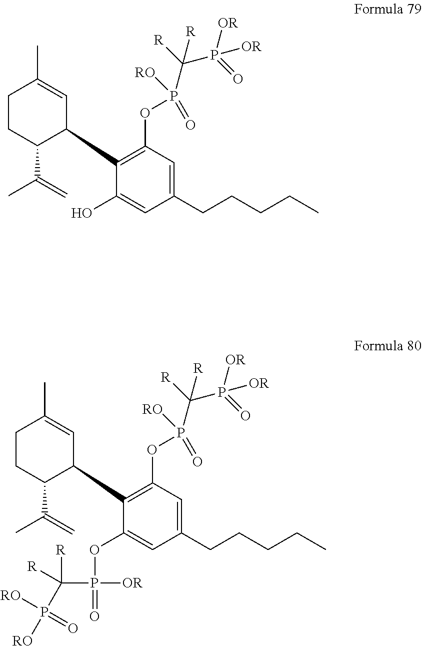

In another embodiment, the cannabinoid compounds are bisphosphonate ester salts of THC represented by the general formula 81:

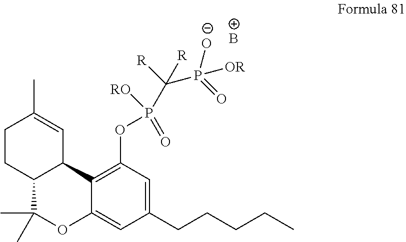

In another embodiment, the cannabinoid compounds are bisphosphonate esters of CBD represented by the general formulas 82 and 83:

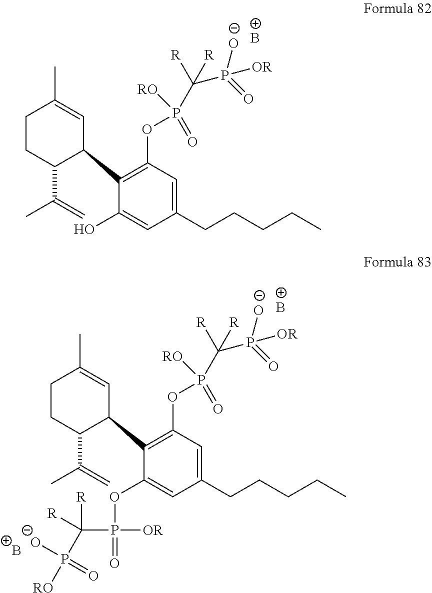

In another embodiment, the second compound, represented by the R group, in formula 1-83, is selected from a group with synergistic or additive effects in combination with the cannabinoid.

In another embodiment, the second compound, represented by R group in formula 1-83, is a second cannabinoid which has a functional group suitable for making a linkage with the first cannabinoid. The functional group may include a thiol, hydroxyl, amino, nitrile, cyanate, isocyanate, thiocyanate, isothiocyanate, azide, carboxylic, acid anhydride, alkene, alkyne, aldehyde, ketone, epoxide, or a phenolic functional group. The second cannabinoid may be selected from natural, synthetic, semisynthetic, biosynthetic, or endogenous cannabinoids. The second cannabinoid may be selected from a group that includes delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM) cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol -C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof.

In another embodiment, the synergistic or additive compound, represented by R group in formula 1-83, has a functional group suitable for making a linkage with the first cannabinoid. The functional group may include a thiol, hydroxyl, amino, nitrile, cyanate, isocyanate, thiocyanate, isothiocyanate, azide, carboxylic, acid anhydride, alkene, alkyne, aldehyde, ketone, epoxide, or a phenolic functional group. The second synergistic or additive therapeutic counterparts are compounds that have a synergistic or additive effect in the treatment of one or more conditions when administered together with the cannabinoid. The second synergistic or additive therapeutic counterparts may be selected from natural, synthetic, semisynthetic, biosynthetic, or endogenous compounds. The second synergistic or additive therapeutic counterparts may be selected from the group that includes glucosamine, psilocybin, psilocin, pregabalin, gabapentin, topiramate, acetaminophen, ibuprofen, morphine, caffeic acid, levodopa, coumaric acid, quercetin, flavonoids, salicylic acid, thymol, eugenol, entacapone, tolcapone, estrogens, selective serotonin reuptake inhibitor (SSRI), androgens and corticosteroids. Preferably, the second synergistic or additive therapeutic counterparts are selected from glucosamine, psilocybin, psilocin, pregabalin, gabapentin, and topiramate.

In another embodiment, the counter ion, represented by group B⁺, in formula 28-83, selected from a group with synergistic or additive effects.

In another embodiment, the counter ion, represented by group B⁺, in formula 28-83, selected from a group that has a functional group suitable for making a salt with the first cannabinoid. B⁺ could be selected from a group that includes cyclic amines, acyclic amines, ethanol amine derivatives, aromatic amines, aliphatic amines, amino sugars, amino polymers, amino oligomers, and amino acids.

In another embodiment, the synergistic or additive compound, represented by B⁺ group in formula 28-83. B⁺ may be selected from the group that includes glucosamine, psilocybin, psilocin, pregabalin, gabapentin, topiramate, morphine, levodopa, selective serotonin reuptake inhibitor (SSRI).

In other embodiments, when the cannabinoid salt has two hydroxy groups, cannabinoid salt can contain two therapeutic agent components. In such embodiments, the two therapeutic agent components can be the same or different.

In another embodiment, certain embodiments of the cannabinoid compounds, according to the present invention, may demonstrate one or more desirable features, including improved stability, higher solubility, higher potency or improved PK or PD properties.

In another embodiment, water-soluble cannabinoid esters, according to the present invention, may be used for edible, beverage, and medicinal applications. The esters may be hemi esters, full esters, mixed esters or salts.

In another embodiment, water-soluble cannabinoid esters, according to the present invention, may be used for topical, injection or oral applications.

In another embodiment, the cannabinoid esters are in the form of water-soluble salts with another synergistic or additive base.

Another embodiment of the present invention is a method of producing a cannabinoid compound by synthetic or semisynthetic methods. The method includes the following steps:
a. Dissolving a cannabinoid having at least one hydroxyl group in a suitable aprotic organic solvent.
b. Reacting the cannabinoid solution with an ester transfer reagent in the presence of an alkali or an organic base.
c. Heating the reaction under conventional heating, microwave heating, or sonication to produce a product.
d. Purify the product using flash chromatography, extraction, distillation, sublimation or crystallization.

In another embodiment, the aprotic organic solvent is selected from the group consisting of pyridine, toluene, tetrahydrofuran, halogenated hydrocarbons, xylenes, and hexanes.

Certain preferred embodiments include sulfate esters and sulfate ester salts, while other embodiments include other types of esters and ester salts. Whether the ester is a sulfate ester or another type of ester or ester salt, an ester transfer reagent may be used that is suitable to the particular type of ester.

In another embodiment, the sulfate ester transfer reagent is selected from a group consisting of free chlorosulfonic acid, protected and free sulfonic acid, protected and free sulfuric acid, sulfur trioxide, sulfur trioxide complexes, sulfur trioxide pyridine, alkali metal disulfate, sulfonyl imidazolium salts, N-hydroxysuccinimide-sulfate and tributylsulfoammonium betaine.

In another embodiment, the carbonate transfer reagents can be selected from phosgene, trichloroacetyl chloride, 1,1′-carbonyldiimidazole (CDI), 1,1′-carbonylbis(2-methylimidazole), N,N′-disuccinimidyl carbonate, 4-nitrophenylchloroformate, bis(4-nitrophenyl)carbonate, bis(pentafluorophenyl)carbonate, and then optionally treated with base, water or alcohol.

In another embodiment, the carbamate transfer reagents can be selected from phosgene, trichloroacetyl chloride, 1,1′-carbonyldiimidazole (CDI), 1,1′-carbonylbis(2-methylimidazole), N,N′-disuccinimidyl carbonate, 4-nitrophenylchloroformate, bis(4-nitrophenyl)carbonate, bis(pentafluorophenyl)carbonate, and then optionally treated with ammonia or, any mono or disubstituted amines.

In another embodiment, the phosphate transfer reagents can be selected from Bis(4-nitrophenyl)phosphate, diphenylphosphate, paraoxon-ethyl, 4-nitrophenylphosphate bis(cyclohexylammonium) salt, 4-nitrophenyl)phosphate sodium salt, 4-nitrophenyl)phosphate sodium salt hydrate, 4-nitrophenyl phosphorodichloridate, diphenyl phosphoryl chloride, diethyl chlorophosphate, dimethyl chlorophosphate or diisopropyl chlorophosphate, diphosphoryl chloride, and then optionally treated with water, ammonia, substituted amines or any alcohol.

In another embodiment, the nitrate transfer reagents can be selected from nitronium tetrafluoroborate, nitrosonium hexafluoroantimonate, nitronium hexafluoroantimonate, sodium nitrite, potassium nitrite, ammonium nitrite, silver nitrite, cadmium nitrite, sodium nitrate, potassium nitrate, ammonium nitrate, silver, nitrate, cadmium nitrate, nitric acid, 1-nitropyrrolidine-2,5-dione, 1-nitropyrrolidin-2-one, 2-nitroisoindoline-1,3-dione, 2-nitrobenzo[d]isothiazol-3(2H)-one 1,1-dioxide, or 2,5-dinitrobenzo[d]isothiazol-3(2H)-one 1,1-dioxide.

In another embodiment, the bisphosphonate transfer reagents can be selected from medronic acid (methylenediphosphonic acid), medronyl chloride, methylenebis(phosphonic dichloride), etidronate, alendronate, ibandronate, minodronate, residronate, tiludronate, zoledronate, esters, hemiesteres of any of the previous reagents.

In another embodiment, the boronate transfer reagents can be selected from boric acid, sodium tetraborate, bis(pinacolato)diboron, (dimethylphenylsilyl)boronic acid pinacol ester, 4,4,5,5-tetramethyl-1,3,2-dioxaborolane, 2-methoxy-4,4,5,5-tetramethyl-1,3,2-dioxaborolane, 2-ethoxy-4,4,5,5-tetramethyl-1,3,2-dioxaborolane, 2-isopropyloxy-4,4,5,5-tetramethyl-1,3,2-dioxaborolane, triisopropyl borate, triethyl borate, trimethyl borate, boron trichloride, chloroborane, chloroborane methyl ester, B-chlorocatecholborane, B-bromocatecholborane, catecholborane, bromodimethylborane, and then optionally treated with water, ammonia, substituted amine, or alcohol.

In another embodiment, various salts and forms of THC sulfate ester salts may be produced according to the method illustrated in Formula 84, below. The pyridine counter ion may be replaced by another selected synergistic or additive bases in quantitative yield (95-99%) and analytical purity (95-98%) as amorphous powder when stirred with 1.2 equiv of the selected base in aqueous solutions and as a crystalline compound in non aqueous solutions. The presence of at least some aqueous solvent in the reaction solution is important to facilitate the replacement of pyridine with the target counter ion. For certain target counter ions with high solubility, the reaction may take place entirely in water. For target counter ions with low solubility, a mixture of aqueous and non-aqueous solvents may be used to facilitate the reaction. Preferably, water and ethanol are used in a 1:1 ratio, but other non-aqueous solvents may also be used.

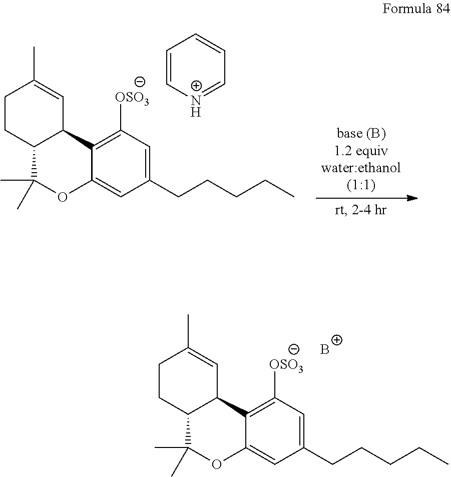

In another embodiment, various salts and forms of CBD sulfate ester salts may be produced according to the method illustrated in Formula 85, below. The pyridine counter ion may be replaced by another selected synergistic or additive bases in quantitative yield (95-99%) and analytical purity (95-98%) as amorphous powder when stirred with 1.2 equiv of the selected base in aqueous solutions and as a crystalline compound in non aqueous solutions.

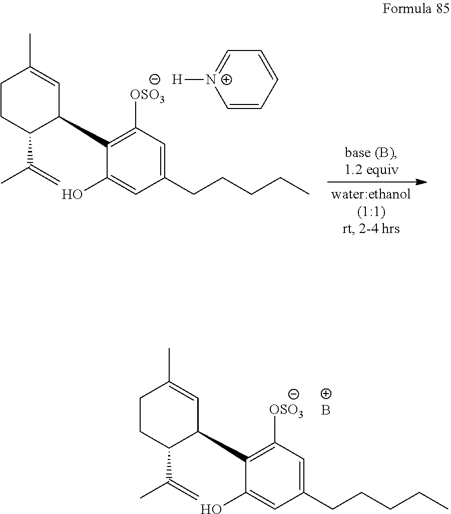

In another embodiment, glucosamine salt of THC sulfate ester salt may be produced according to a one pot, two step reaction sequence illustrated in Formula 86, below, to obtain the target sulfate in quantitative yield (90-99%) and analytical purity (95-98%). The temperature is between 65-90° C., the pressure is between 5-20 bar, and the reaction time is between 4-8 hrs in pyridine. The first step of the method of Formula 86 is to convert THC into a THC sulfate ester salt by mixing the THC with pyridine sulfur trioxide in pyridine at 65-90° C. and between 5-20 bar pressure for 2-4 hours. The reaction is highly efficient, utilizing safe and easy to handle reagents, providing the target sulfate ester salt in a quantitative yield and analytical purity not less than 95%. The second step is the counter ion replacement, as described in Formulas 84 and 85, with glucosamine as the counter ion. No purification step is required between the first and second steps and they can be carried out immediately in sequence in the same reaction vessel. Alternatively, the reaction may be carried out in a parallel reaction, whereby the first step is carried out in a large-scale reaction and the crude product is split into a plurality of separate reactions to carry out the second step with different counter ions.

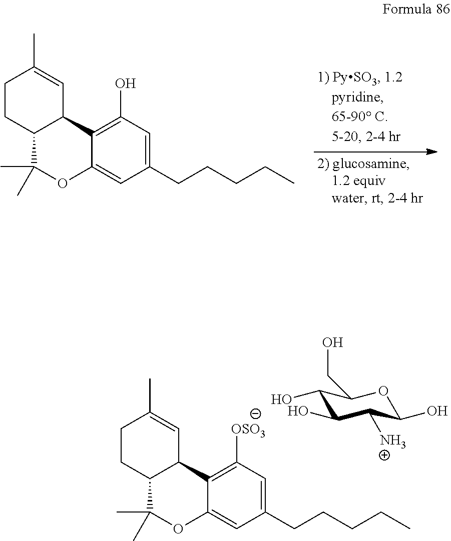

In another embodiment, glucosamine salt of CBD sulfate ester may be produced according to the method illustrated in Formula 87, below, to obtain the target sulfate in quantitative yield (90-99%) and analytical purity (95-98%). The temperature is between 65-90° C., the pressure is between 5-20 bar, and the reaction time is between 4-8 hrs in pyridine.

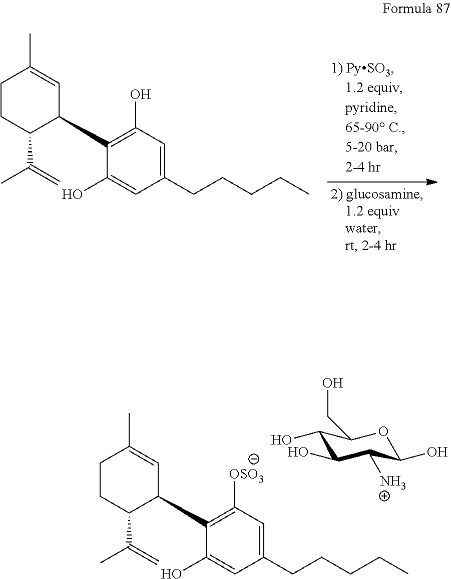

In another embodiment, psilocin salt of THC sulfate ester salt may be produced according to the method illustrated in Formula 88, below, to obtain the target sulfate in quantitative yield (90-99%) and analytical purity (95-98%). The temperature is between 65-90° C., the pressure is between 5-20 bar, and the reaction time is between 4-8 hrs in pyridine.

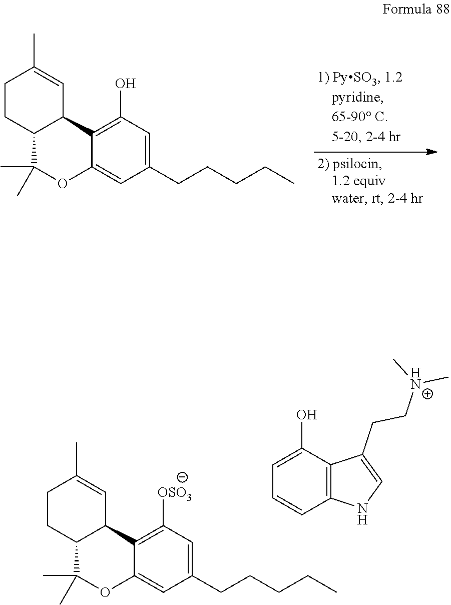

In another embodiment, psilocin salt of CBD sulfate ester may be produced according

to the method illustrated in Formula 89, below, to obtain the target sulfate in quantitative yield (90-99%) and analytical purity (95-98%). The temperature is between 65-90° C., the pressure is between 5-20 bar, and the reaction time is between 4-8 hrs in pyridine.

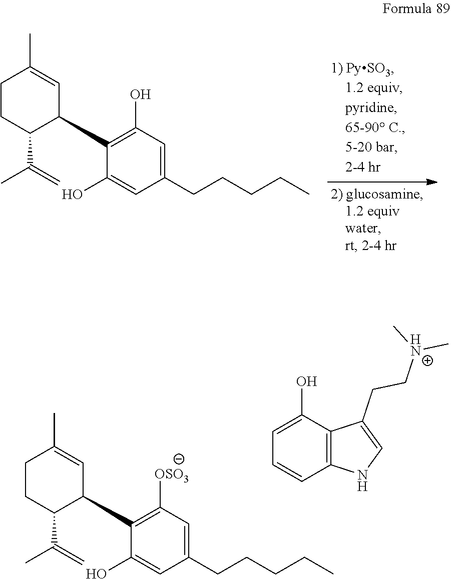

In another embodiment, glucosamine salt of THC phosphate ester salt may be

produced according to the method illustrated in Formula 90, below, to obtain the target phosphate in quantitative yield (90-99%) and analytical purity (95-98%). The temperature is about 50° C., the pressure is between 5-20 bar, and the reaction time is between 4-8 hrs in tetrahydrofuran (THF).

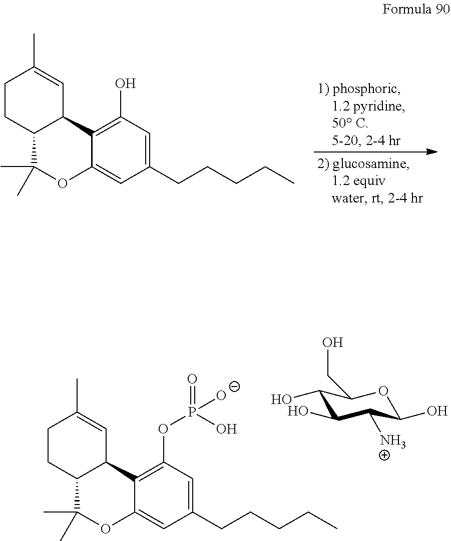

In another embodiment, glucosamine salt of CBD phosphate ester may be produced according to the method illustrated in Formula 91, below, to obtain the target phosphate in quantitative yield (90-99%) and analytical purity (95-98%). The temperature is about 50° C., the pressure is between 5-20 bar, and the reaction time is between 4-8 hrs in tetrahydrofuran (THF).

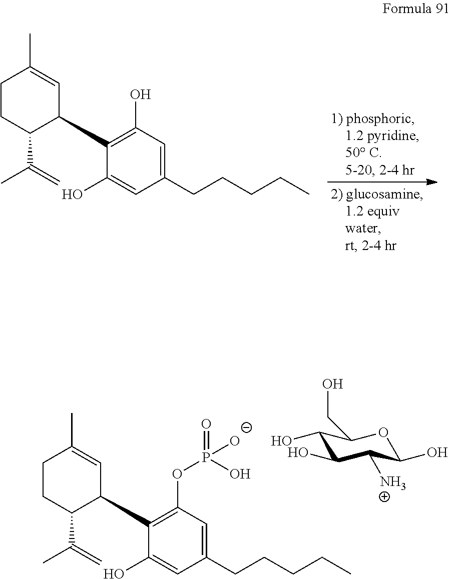

In another embodiment, psilocin salt of THC phosphate ester salt may be produced according to the method illustrated in Formula 92, below, to obtain the target phosphate in quantitative yield (90-99%) and analytical purity (95-98%). The temperature is about 50° C., the pressure is between 5-20 bar, and the reaction time is between 4-8 hrs in tetrahydrofuran (THF).

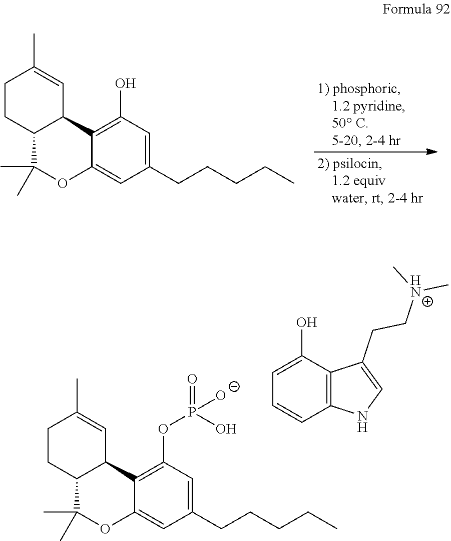

In another embodiment, psilocin salt of CBD phosphate ester may be produced according to the method illustrated in Formula 93, below, to obtain the target phosphate in quantitative yield (90-99%) and analytical purity (95-98%). The temperature is about 50° C., the pressure is between 5-20 bar, and the reaction time is between 4-8 hrs in tetrahydrofuran (THF).

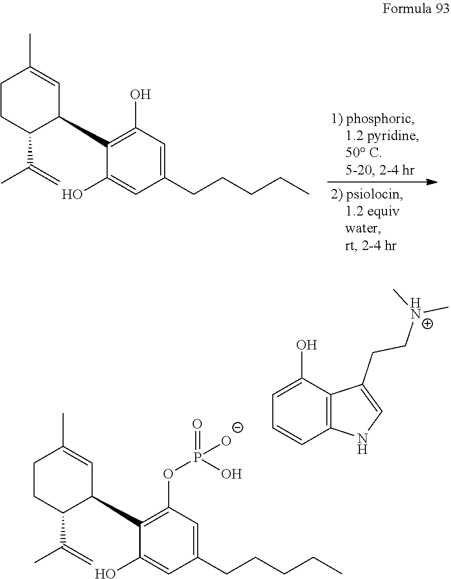

In certain embodiments, the cannabinoid esters may act on either or both peripheral and central tissues.

In another embodiment, the cannabinoid esters are peripherally restricted, such that they lack the central psychoactive properties of THC.

In another embodiment, the cannabinoid esters may be used to manage several conditions including pain and inflammation, mental health disorders, cancer, glaucoma, neurodegenerative disorders, multiple sclerosis, renal fibrosis, fibrotic disorder, addiction, motor function disorders and gastrointestinal and metabolic disorders and other conditions that respond to cannabinoid receptor modulation or are otherwise known to be treatable by administration of one or more cannabinoids.

In another embodiment, the cannabinoid esters may be used for both human and animal applications.

In certain embodiments, the cannabinoid esters are particularly useful for oral delivery systems. In addition, they may be formulated for topical, intranasal, ophthalmic or parenteral delivery systems.

In another embodiment, the cannabinoid esters include all possible isomers (stereo or structural) either as individual hemi esters, full esters, salts or mixtures thereof.

In another embodiment, pharmaceutical formulations of cannabinoid esters, according to the present invention, include other synergistic or additive ingredients including other cannabinoids, phytochemicals, analgesics and anti-inflammatories.

When compared to other cannabinoids, the present invention discloses cannabinoid compounds with improved PK and PD profiles, including better stability, solubility and taste, efficient absorption and distribution, and potency, which may provide effective disease control and therapeutic effects.

### DESCRIPTION OF THE INVENTION

This disclosure relates to cannabinoid compounds, in particular, cannabinoid esters that can act as cannabinoid drugs or prodrugs, to methods of producing cannabinoid esters, and their salts with synergistic or additive therapeutic counterparts, to edible, beverage, and pharmaceutical formulations of these compounds, to methods of modulating the endocannabinoid system by administering cannabinoid esters to a patient, and to methods of treating pain, neuropathic pain, inflammation, neurodegenerative disorders, multiple sclerosis, spinal cord and brain injury, post-traumatic stress disorder, epilepsy and other motor disfunctions, paediatric seizure disorders, addiction, insomnia, nausea and vomiting, cancer, renal fibrosis, obesity and other metabolic disorders, schizophrenia, depression, obsessive compulsive disorders, anxiety, psychiatric disorders, sleep disorders, fibromyalgia, Tourette syndrome, glaucoma, Crohn's disease, inflammatory bowel disorders, cluster headache, anorexia and other conditions by administering cannabinoid esters to a patient.

The cannabinoid esters, according to the present invention, or their active metabolites may act as ligands for either or both CB1 or CB2 or exert their actions through a non-receptor mediated mechanism(s). Due to the unique pharmacokinetics of certain exemplary embodiments of the present invention, some embodiments may be used as biased modulators (agonists, antagonists, partial agonists, inverse agonists, etc.) to selectively bind to a first cannabinoid receptor over a second cannabinoid receptor, such as CB1, CB2, or any other endocannabinoid receptors in a subject. They may also modulate other targets and receptors including COX enzymes, fatty acid amide hydrolase (FAAH), transient receptor potential cation channel subfamily V (TrpV), peroxisome proliferator-activated receptors, putative abnormal-cannabidiol receptor, ion channels, ligand gated ion channels and other G-protein coupled receptors.

Compounds according to certain exemplary embodiments of the present invention have shown 2,000-5,000 fold increased water solubility, compared to the base cannabinoid compound. Certain exemplary compounds have also shown good stability under various pH conditions. While these exemplary compounds are hydrolyzed quickly under pH 1.2 (fasting simulated gastric fluid), they show good stability under pH 5.1, 6.8, and 7.4 (fasting simulated intestinal fluid). Furthermore, certain exemplary compounds has shown that both aryl sulfatase and b-glucoronidase are able to break down about 50% of the initial amount of the compound within 6 hours. In rat pharmacokinetic studies, certain exemplary compounds have shown a 10-fold increase in absorption and relative bioavailability with oral dosage, permitting the use of 1/10^{th} the regular dose of the cannabinoid, and are suitable for once-daily dosing regimes.

The term “esters” includes all possible hemiesters, full esters, salts and isomers, including, stereoisomers, enantiomers, diastereomers, tautomers, and mixtures, by any ratio(s), thereof. Preferably, the esters are hemiesters or salts. Preferably, they are salts of pure compounds.

The term “cannabinoid” relates to a cannabinoid with at least one hydroxyl group. It includes endogenous, synthetic, semisynthetic, or natural cannabinoids, including: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM) cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof.

The terms “hydroxyl” group relates to alcoholic or phenolic OH or their isosteres (e.g., SH or NH₂).

The term “salts” refers to salts with synergistic or additive therapeutic counterparts. The term “salts” also refers to salts of organic bases with pKa more than 3, including: cyclic or acyclic amines (e.g. erbumine), ethanol amine derivatives (triethanol amine), basic amino acids (e.g. arginine, lysine), amino sugar (e.g. glucosamine), amino polymers and oligomers (deacetylated chondroitin, deacetylated hyaluronic acid), aromatic or aliphatic amines (e.g. aniline, 4-aminopyrimidine) or other cyclic nitrogen compounds (e.g. aziridine, azetidine, diazetidine, imidazoline, pyrazolidine, 3-pyrroline, triazole, imidazole, pyrrolidine, piperidine, pyridine, piperazine, pyridazine, pyrimidine, pyrazine, morpholine, thiomorpholine dioxide, thiazine, pyrrolizidine, azaindole, azaindazole, purine, pyrazolo pyrimidine, quinoline, decahydroquinoline, azocane), psilocybin, psilocin, pregabalin, gabapentin, topiramate, morphine, levodopa, selective serotonin reuptake inhibitor (SSRI).

The term “pro-drug” is intended to include esters of the target compounds that may require activation within the human body. The esters or their salts may be active (equipotent or more potent) or inactive compounds. Preferably, they are active. Upon administration to human or animal subjects, they undergo enzymatic or chemical activation to release the free drug.

The term “pharmaceutical formulation”, as used herein, refers to a mixture of one or more of the compounds described herein, or pharmaceutically acceptable salts thereof, or other synergistic or additive therapeutic counterparts along with other physiologically acceptable carriers and excipients. The purpose of a pharmaceutical formulation (e.g. solid or liquid dosage forms) is to facilitate administration of a compound to a subject animal or human.

The term “subject” in the present disclosure refers to human patients but is not limited to humans and may include animals.

As used herein, the term “administering” includes all means of introducing the compounds and compositions described herein to the patient, including, but are not limited to, oral, intravenous, intramuscular, transdermal, inhalation, buccal, ocular, vaginal, rectal, and the like. The compounds and compositions described herein may be administered in unit dosage forms and/or formulations containing conventional nontoxic pharmaceutically acceptable carriers, adjuvants, and vehicles.

In a preferred embodiment, the cannabinoid esters of the present invention are represented by the examples in Formula 28, 29, 32, 34, 61, 62, 69 and 71. Preferably, the cannabinoid esters are in the form of salts with synergistic or additive therapeutic counterparts. Preferably, salts of glucosamine, psilocybin, psilocin, pregabalin, gabapentin, topiramate, morphine, levodopa, selective serotonin reuptake inhibitor (SSRI) (e.g., citalopram), amisulpride, lurasidone, paliperidone, paliperidone palmitate, risperidone, ziprasidone, perospirone, doxorubicin, melperone, aripiprazole, brexpiprazole, cariprazine, olanzapine, quetiapine, flouxetine, calcitonin, pseudoephedrine, piracetam, levetiracetam, sitagliptin, silodosin, hydrochlorothiazide, ezetimibe, propranolol, atenolol, nadolol, pindolol, sotalol, timolol, penbutolol, oxprenolol, carvidiol, carteolol, bucindolol, acebutanol, betaxolol, esmolol, nebivolol, bisoprolol, celiprolol, metorpolol, azelnidipine, barnidipine, manidipine, lercandipine, efonidipine, benidipine, brimonidine, bortezomib, ledipasvir, daclatasvir, ombitasvir, elbasvir, lamivudine, dopamine, 5-hydroxytryptamine, levodopa, pramipexole, ropinirole, rotigotine, apomorphine, tacrine, rivastigmine, donepezil, galantamine, vigabatrin, lamotrigine, tiagabine, pregabalin, amitriptyline, nortriptyline and histamine.

In another preferred embodiment, the cannabinoid esters of the present invention are represented by the examples in Formula 28, 29, 32, 34, 61, 62, 69 and 71. Preferably, the cannabinoid esters are in the form of salts with synergistic or additive therapeutic counterparts. Preferably, salts of opiate receptor antagonists such as loperamide and diphenoxylate; opiate receptor agonists such as tapentadol, or those with mixed agonist-antagonist and/or partial agonist effect of opiate receptor(s) such as nalbuphine, buprenorphine and pentazocine. These preferred embodiments are illustrated in Table 2 below, where the base (B) may be any of the bases a-h listed in Table 3 below.

TABLE 2
Exemplary cannabinoid ester salts.

| | |
|---|---|
| 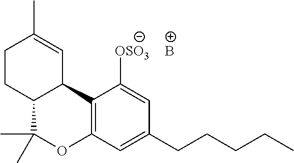 | Formula 28 |
| 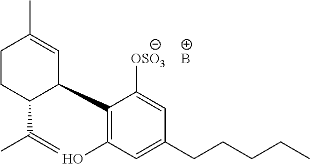 | Formula 29 |
| 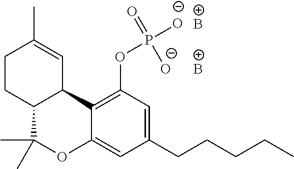 | Formula 32 |
| 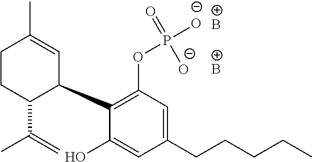 | Formula 34 |
| 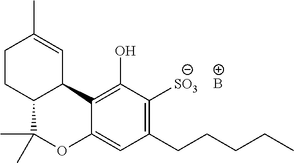 | Formula 61 |
| 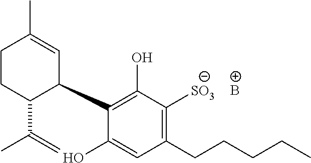 | Formula 62 |
| 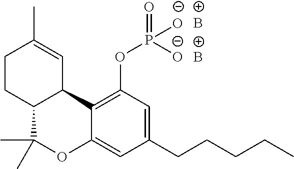 | Formula 69 |
| 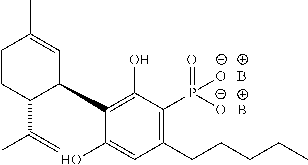 | Formula 71 |

TABLE 3
Exemplary bases.

| entry | Base (B) |
|---|---|
| a | glucosamine |
| b | psilocin |
| c | pregabalin |
| d | gabapentin |
| e | topiramate |
| f | morphine |
| g | levodopa |
| h | citalopram |

The side groups R in formulas 1 to 83 may be another cannabinoid, other active ingredients, or inactive groups. Preferably, R is another compound with synergistic or additive activity. The other cannabinoid is preferably THC or CBD, but may be any other cannabinoid with a hydroxyl, amino, or phenolic functional group. Examples of suitable cannabinoids include, cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM) cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC -C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. The other active ingredient is preferably acetaminophen or ibuprofen, but may include opioids or other medications with at least one hydroxyl, amino, or phenolic functional group. The inactive group is preferably H, but may be methyl, ethyl, or another acyclic saturated hydrocarbon group (i.e. CₙH₂ₙ₊₁), aryl or another cyclic saturated hydrocarbon group (i.e. CₙH₂ₙ₋₁), or their isosteres and analogues.

The counter ion, also referred to herein as the base, represented by B⁺ in Formulas 28 to 83, may be a cyclic amine, acyclic amine, ethanol amine derivative, aromatic amine, aliphatic amine, amino sugar, amino polymer, amino oligomer, or amino acid. Preferably, it is triethanol amine, erbumine, arginine, or lysine, but may be, ammonia, triethyl amine, trimethyl amine, tripropyl amine, tributyl amine, and other related amines and derivatives including primary, secondary, and tertiary. The aromatic amine is preferably aniline or 4-aminopyrimidine, but may be naphthylamine, sulfanilic acid, 4-amino benzoic acid, and other related amines, analogues, and derivatives. The side group may also preferably be piperazine or morpholine, but may be aziridine, azetidine, diazetidine, imidazoline, pyrazolidine, 3-pyrroline, triazole, imidazole, pyrrolidine, piperidine, pyridine, pyridazine, pyrimidine, pyrazine, thiomorpholine dioxide, thiazine, pyrrolizidine, azaindole, azaindazole, purine, pyrazolo pyrimidine, quinoline, decahydroquinoline, azocane, or their derivatives, analogues, and isosteres.

The therapeutic ion, represented by B⁺ group in formula 28-83, is preferably selected from a group with synergistic or additive effects. Preferably, it is glucosamine, psilocybin, psilocin, pregabalin, gabapentin, topiramate, morphine, levodopa, or a selective serotonin reuptake inhibitor (SSRI) (e.g. citalopram).

The cannabinoid esters can be prepared by synthetic, semisynthetic, microbial, enzymatic and synthetic biology methods, as well as by genetic manipulation of Cannabis sp. Preferably, they can be prepared according to the reactions described in Formulas 84-93, from any cannabinoid with at least one hydroxyl group. Preferably, the cannabinoid is THC or CBD, and the hydroxyl group is a phenolic OH. Modification of the reaction condition(s) can produce other derivatives and analogues.

Pharmaceutical formulations may be prepared including the cannabinoid esters or any pharmaceutically acceptable salts thereof, and one or more pharmaceutically acceptable carriers or excipients. Preferably the formulation is a solid or liquid dosage form for oral and oromucosal applications.

The pharmacokinetic profile of certain exemplary embodiments of the cannabinoid esters is more favourable than the corresponding parent cannabinoids. For example, the Cₘₐₓ (maximum plasma concentration) of certain exemplary glucosamine cannabinoid sulfate salts was 10-fold higher than the parent cannabinoids. As a result, pharmaceutical formulations may contain lower effective doses of these cannabinoid sulfate ester salts, as compared to the parent cannabinoids. In addition, exemplary cannabinoid sulfate ester salts have less variable absorption than the parent cannabinoids. The PK profile of certain exemplary cannabinoid sulfate esters, including the half-life (T_{1/2}), maximum plasma concentration (Cₘₐₓ), and time to reach Cₘₐₓ (Tₘₐₓ), is shown compared to CBD in the table below.

| | | Glucosamine CBD |
|---|---|---|
| | CBD | sulfate salt |
| T_{1/2} (h) | 3.9 | 1.7 |
| Cmax | 4.8 | 44.0 |
| (ng/ml) | | |
| Tmax (h) | 0.5 | 1 |

The enzymatic and chemical stability of certain exemplary embodiments of the ester salts under simulated stomach and intestinal media is more favourable than the corresponding parent cannabinoids. For example, CBD can be released within a short time under simulated stomach and intestinal media (5% to 20% released within 10 to 30 minutes). Exemplary cannabinoid sulfate ester salts also show favourable toxicity profiles compared to the corresponding parent cannabinoids. Further, certain exemplary cannabinoid sulfate ester salts show an aqueous solubility of 5,000 to 30,000-fold higher than the parent cannabinoids.

The formulation may also contain synergistic or additive ingredients, in addition to active ingredients, which may include: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM) cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof Boswellia sp., including Boswellia carterii and Boswellia serrata, ginger, capsaicin, camphor, polyphenols, including quercetin, ellagic acid, curcumin, and resveratrol, phytosterols, carbohydrates, including mannose-6-phosphate; essential oils, including thymol, and carvacrol, terpenoids, including squalene, lycopene, p-cymene, linalool, and derivatives and analogues thereof, or mixtures or combinations thereof. Preferably, the formulation contains additional synergistic or additive ingredient, to the selected cannabinoid ester compound(s).

The designed compounds, according to the present invention, can be delivered by oromucosal, nasal, oral, ophthalmic, transdermal and parenteral routes. Preferably, they are delivered by oral routes or transdermal.

The cannabinoid esters, according to the present invention, may be used in various

applications, including edibles, beverages and medical applications. Preferably, they may be used for the treatment of inflammation and pain, mental health disorders, and other related conditions that respond to modulation of cannabinoid receptors. Compared to some other related analogues, preferred embodiments of the salts of these esters are more stable and water soluble with improved absorption, as well as optimized pharmacokinetic and pharmacodynamic profiles. They may be useful in the treatment of inflammation, pain, mental health disorders and related conditions to quickly alleviate the symptoms and provide long-lasting relief to the patient.

### EXAMPLES

### Example 1

### Preparation of Pyridine Salt of CBD Sulfate Ester

A reaction tube with a rubber cap, Teflon septum and stir bar is charged with cannabidiol (CBD) (1.58 g, 5 mmol, 1 equiv), Py.SO3 (97%) (0.96 g, 6 mmol, 1.2 equiv) and 3 mL dry pyridine. The reaction tube is flushed with argon gas and heated at 70° C. for 4 hr, under a pressure of 5-20 barr in a Monowave 50® by Anton Paar. After cooling to room temperature, pyridine is evaporated at reduced pressure (100 mbar) and 50° C. to give the desired product as a viscous oil (2.36 g, quantitative yield). Optionally, the product may be used directly in the method of example 2, below, without purification. When other solvents (such as dichloromethane or tetrahydrofuran), room temperature, or atmospheric pressure were utilized, lower yields were obtained (<50%). When higher temperatures (>100° C.) were used, decompositions were observed.

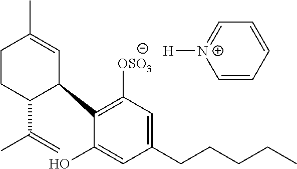

pyridin-1-ium (1′R,2′R)-6-hydroxy-5′-methyl-4-pentyl-2′-(prop-1-en-2-yl)-1′,2′,3′,4′-tetrahydro-[1,1′-biphenyl]-2-yl sulfate:

¹H NMR (60 MHz, CD₃OD) δ ppm 8.79 (d, J=5.49 Hz, 2 H), 8.50 (d, J=7.94 Hz, 1 H), 8.01 (t, J=6.56 Hz, 2 H), 6.08-7.16 (m, 2 H), 5.30 (br. s., 1 H), 4.44 (br. s., 2 H), 3.75-4.22 (m, 1 H), 2.67-3.15 (m, 1 H), 1.77-2.57 (m, 6 H), 1.64 (s, 6 H), 1.34 (br. s., 6 H), 0.71-1.01 (m, 3 H); ¹³ C NMR δ 157.67, 153.34, 150.34, 146.52, 144.41, 142.62, 133.57, 128.31, 127.02, 118.17, 113.92, 111.00, 110.65, 46.42, 38.50, 36.63, 32.77, 32.03, 31.80, 30.82, 23.88, 23.69, 19.55, 14.52; HRMS m/z for C₂₁H₂₉O₅S⁻, calculated: 393.1741, found: 393.1740.

### Example 2

### General Procedures for Counter Ion Exchange

Optionally, the pyridine counter ion of the product of the method of example 1, above, may be replaced by other selected bases in quantitative yield (95-99%) and analytical purity (95-98%) as amorphous powder when stirred with 1.2 equiv of the selected base (e.g. glucosamine or psilocin) in aqueous solution, according to the following method. The aqueous solution may be 1:1 mixture of ethanol:water, methanol:water, pyridine:water, and/or isopropanol:water. Alternatively, water may be mixed with other organic solvents such as acetone, THF, or chloroform. When the counter ion exchange reactions runs in non aqueous solutions (e.g., absolute ethanol or methanol, or dry pyridine), the target sulfates were obtained in crystalline form.

A reaction vial with polyethylene plug and stir bar is charged with pyridinium CBD sulfate, which is preferably produced according to the method of example 1 (0.47 g, 1 mmol, 1 equiv), a selected base (1.2 mmol, 1.2 equiv) and 5 mL of H2O or ethanol or H2O:ethanol solution (1:1). The reaction is stirred at rt for 2-4 hr to produce a milky emulsion which is cooled down to −80° C., and the solvents are freeze-dried, preferably using FreeZone® 2.5 Liter Benchtop Freeze, to give the desired product as amorphous powder in quantitative yields without the need for further purification. The following exemplary CBD sulfate ester salts may be produced according to the method of example 2, by selecting the appropriate base to mix with the pyridinium CBD sulfate in solution.

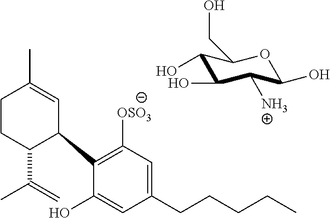

(2R,3R,4R,5S,6R)-2,4,5-trihydroxy-6-(hydroxymethyl)tetrahydro-2H-pyran-3-aminium (1′R,2′R)-6-hydroxy-5′-methyl-4 pentyl-2′-(prop-1-en-2-yl)-1′,2′,3′,4′-tetrahydro-[1,1′-biphenyl]-2-yl sulfate:

¹H NMR (60 MHz, CD₃OD) δ ppm 6.13-7.18 (m, 2 H), 5.39 (d, J=3.36 Hz, 2 H), 4.43 (s, 2 H), 3.87-4.01 (m, 1 H), 3.71-3.84 (m, 4 H), 3.34-3.36 (m, 1 H), 3.06-3.22 (m, 1 H), 2.87-3.05 (m, 1 H), 1.68-2.84 (m, 6 H), 1.64 (s, 6 H), 1.28-1.37 (m, 6 H), 0.78-0.96 (m, 3 H); ¹³C NMR δ 157.28, 152.99, 150.34, 142.78, 134.39, 126.71, 117.98, 113.76, 111.85, 111.00, 90.76, 73.33, 71.61, 71.42, 62.17, 56.17, 46.42, 38.70, 36.55, 32.62, 31.88, 31.68, 30.55, 23.88, 23.57, 19.47, 14.52; HRMS m/z for C₂₁H₂₉O₅S⁻, calculated: 393.1741, found: 393.1740.

Other exemplary embodiments of the present invention are pharmaceutical compositions for treating patients suffering from conditions or diseases that are known to respond to treatment by cannabinoids. These pharmaceutical compositions comprise a compound disclosed herein, or a pharmaceutically acceptable salt thereof, together with one or more pharmaceutically acceptable diluents or excipients, or both.

### Example 3

Each hard gelatin capsule contains:

| Ingredient | Quantity (mg) | Function |
|---|---|---|
| Glucosamine CBD-sulfate | 25 | Active |
| Glucosamine sulfate | 750 | Active |
| Microcrystalline cellulose pH102 | 315 | Diluent |
| Magnesium stearate | 30 | Lubricant |
| Silicon dioxide | 30 | Glidant |

### Example 4

Each hard gelatin capsule contains:

| Ingredient | Quantity (mg) | Function |
|---|---|---|
| Glucosamine CBD-sulfate | 25 | Active |
| Glucosamine sulfate | 750 | Active |
| Chondroitin sulfate | 600 | Active |
| Methylsulfonylmethane (MSM) | 300 | Active |
| Microcrystalline cellulose pH102 | 140 | Diluent |
| Magnesium stearate | 30 | Lubricant |
| Silicon dioxide | 30 | Glidant |

### Example 5

Each hard gelatin capsule contains:

| Ingredient | Quantity (mg) | Function |
|---|---|---|
| Glucosamine CBD-sulfate | 25 | Active |
| Glucosamine sulfate | 750 | Active |
| Chondroitin sulfate | 600 | Active |
| Microcrystalline cellulose pH101 | 140 | Diluent |
| Povidone K30 | 60 | Binder |
| Croscarmellose sodium | 60 | Disintegrant |
| Magnesium stearate | 30 | Lubricant |
| Silicon dioxide | 30 | Glidant |

### Example 6

Each hard gelatin capsule contains:

| Ingredient | Quantity (mg) | Function |
|---|---|---|
| Glucosamine CBD-sulfate | 25 | Active |
| Glucosamine sulfate | 750 | Active |
| Chondroitin sulfate | 600 | Active |
| Methylsulfonylmethane (MSM) | 300 | Active |
| Collagen | 200 | Active |
| Microcrystalline cellulose pH102 | 140 | Diluent |
| Magnesium stearate | 30 | Lubricant |
| Silicon dioxide | 30 | Glidant |

### Example 7

Each tablet contains:

| Ingredient | Quantity (mg) | Function |
|---|---|---|
| Glucosamine CBD-sulfate | 25 | Active |
| Pregelatinized starch | 120 | Diluent |
| Mannitol | 300 | Diluent |
| Hydroxylpropyl methylcellulose EF | 15 | Binder |
| Copovidone | 20 | Disintegrant |
| Talc | 5 | Lubricant |
| Silicon dioxide | 5 | Glidant |

### Example 8

Each 100 g cream contains the following formula

| Ingredient | Quantity (mg) | Function |
|---|---|---|
| Histamine CBD-sulfate | 750 | Active |
| Glucosamine sulfate | 2500 | Active |
| Sorbitan monostearate | 0.75 | Emulsifier |
| Tween 60 | 3 | Surfactant |
| Cetostearyl alcohol | 6 | Stabilizer |
| Propylene glycol | 5 | solvent |
| Benzyl alcohol | 1 | Co- solvent |
| Methyl paraben | 0.14 | Preservative |
| BHT (Butylated hydroxytoluene) | 0.02 | Anti-oxidant |
| Medium chain triglycerides | 8 | Cream Base |
| Isopropyl myristate | 5 | emollient |
| Purified water | to 100 | Vehicle |

The term “pharmaceutically acceptable diluent” or “pharmaceutically acceptable excipient” refers to a pharmaceutically-acceptable material, composition or vehicle, such as a liquid or solid filler, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include : (1) sugars, such as lactose and maltose; (2) starches, such as corn starch and gelatinized starch; (3) cellulose, and its derivatives, such as carboxymethyl cellulose salt, and hydroxypropylmethyl cellulose; (4) thickening agents such as gelatin and tragacanth; (5) disintegrants such as copovidone; (6) other excipients, such as cocoa butter and suppository waxes and pyrogen-free water for sterile products; and (7) other non-toxic compatible substances employed in pharmaceutical formulations.

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A cannabinoid compound, comprising a labile ester of a cannabinoid with a synergistic or additive therapeutic counterpart.

2. The cannabinoid compound of claim 1, wherein the labile ester is an ester selected from the group consisting of: sulfate, hemisulfate, mono-phosphate, di-phosphate, tri-phosphate, carbonate, carbamate, nitrate, borate, sulfonate, phosphonate, and bisphosphonate.

3. The cannabinoid compound of claim 2, wherein the cannabinoid is selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM) cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH).

4. The cannabinoid compound of claim 3, wherein the therapeutic counterpart is a second cannabinoid which has a functional group suitable for making a labile ester linkage with the cannabinoid.

5. The cannabinoid compound of claim 4, wherein the functional group is selected from the group consisting of: thiol, hydroxyl, amino, nitrile, cyanate, isocyanate, thiocyanate, isothiocyanate, azide, carboxylic, acid anhydride, alkene, alkyne, aldehyde, ketone, epoxide, and phenolic.

6. The cannabinoid compound of claim 3, wherein the therapeutic counterpart is selected from the group consisting of: glucosamine, psilocybin, psilocin, pregabalin, gabapentin, topiramate, acetaminophen, ibuprofen, morphine, caffeic acid, levodopa, coumaric acid, quercetin, flavonoids, salicylic acid, thymol, eugenol, entacapone, tolcapone, an estrogen, a selective serotonin reuptake inhibitor (SSRI), an androgen, and a corticosteroid.

7. The cannabinoid compound of claim 3, wherein the labile ester is an ester salt and the therapeutic counterpart is a counter ion of the ester salt and has a functional group suitable for making an ester salt with the cannabinoid.

8. The cannabinoid compound of claim 7, wherein the counter ion is a primary, secondary, or tertiary amine selected from the group consisting of: a cyclic amine, an acyclic amine, an ethanol amine derivative, an aromatic amine, an aliphatic amine, an amino sugar, an amino polymer, an amino oligomer, and an amino acid.

9. The cannabinoid compound of claim 8, wherein the counter ion is selected from the group consisting of: triethanol amine, erbumine, arginine, lysine, ammonia, triethyl amine, trimethyl amine, tripropyl amine, and tributyl amine.

10. The cannabinoid compound of claim 8, wherein the counter ion is an aromatic amine selected from the group consisting of: aniline, 4-aminopyrimidine, naphthylamine, sulfanilic acid, and 4-amino benzoic acid.

11. The cannabinoid compound of claim 7, wherein the counter ion is selected from the group consisting of: piperazine, morpholine, aziridine, azetidine, diazetidine, imidazoline, pyrazolidine, 3-pyrroline, triazole, imidazole, pyrrolidine, piperidine, pyridine, pyridazine, pyrimidine, pyrazine, thiomorpholine dioxide, thiazine, pyrrolizidine, azaindole, azaindazole, purine, pyrazolo pyrimidine, quinoline, decahydroquinoline, and azocane.

12. The cannabinoid compound of claim 7, wherein the counter ion is selected from the group consisting of: glucosamine, psilocybin, psilocin, pregabalin, gabapentin, topiramate, morphine, levodopa, selective serotonin reuptake inhibitor (SSRI) (e.g., citalopram), amisulpride, lurasidone, paliperidone, paliperidone palmitate, risperidone, ziprasidone, perospirone, doxorubicin, melperone, aripiprazole, brexpiprazole, cariprazine, olanzapine, quetiapine, flouxetine, calcitonin, pseudoephedrine, piracetam, levetiracetam, sitagliptin, silodosin, hydrochlorothiazide, ezetimibe, propranolol, atenolol, nadolol, pindolol, sotalol, timolol, penbutolol, oxprenolol, carvidiol, carteolol, bucindolol, acebutanol, betaxolol, esmolol, nebivolol, bisoprolol, celiprolol, metorpolol, azelnidipine, barnidipine, manidipine, lercandipine, efonidipine, benidipine, brimonidine, bortezomib, ledipasvir, daclatasvir, ombitasvir, elbasvir, lamivudine, dopamine, 5-hydroxytryptamine, levodopa, pramipexole, ropinirole, rotigotine, apomorphine, tacrine, rivastigmine, donepezil, galantamine, vigabatrin, lamotrigine, tiagabine, pregabalin, amitriptyline, nortriptyline and histamine.

13. The cannabinoid compound of claim 2, wherein the cannabinoid compound is a compound of formula 1: 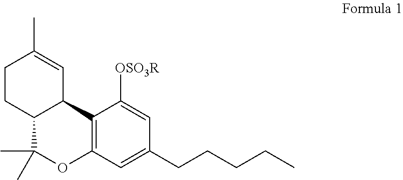
wherein R is the therapeutic counterpart.

14. The cannabinoid compound of claim 2, wherein the cannabinoid compound is a compound of formula 2: 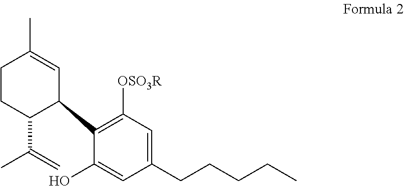
wherein R is the therapeutic counterpart.

15. The cannabinoid compound of claim 2, wherein the cannabinoid compound is a compound of formula 3: 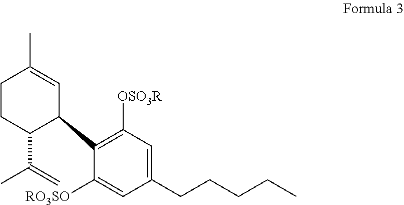
wherein each R is the therapeutic counterpart, selected independently.

16. The cannabinoid compound of claim 2, wherein the cannabinoid compound is a compound of formula 4: 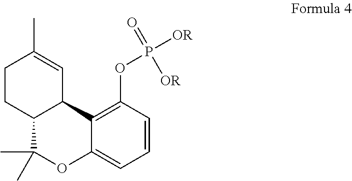
wherein each R is the therapeutic counterpart, selected independently.

17. The cannabinoid compound of claim 2, wherein the cannabinoid compound is a compound of formula 5: 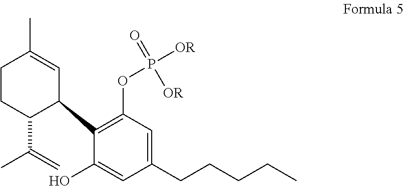
wherein each R is the therapeutic counterpart, selected independently.

18. The cannabinoid compound of claim 2, wherein the cannabinoid compound is a compound of formula 6: 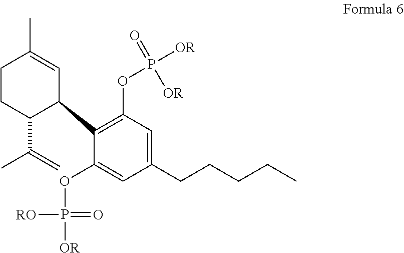
wherein each R is the therapeutic counterpart, selected independently.

19. The cannabinoid compound of claim 2, wherein the cannabinoid compound is a compound of formula 7: 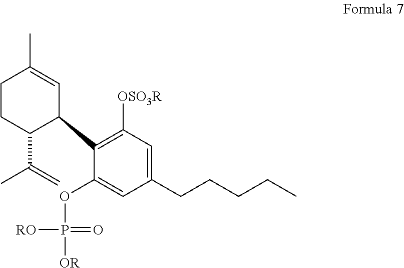
wherein each R is the therapeutic counterpart, selected independently.

20. The cannabinoid compound of claim 2, wherein the cannabinoid compound is a compound of formula 8: 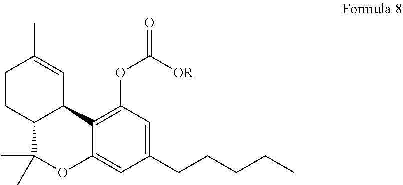
wherein R is the therapeutic counterpart.

21. The cannabinoid compound of claim 2, wherein the cannabinoid compound is a compound of formula 9: 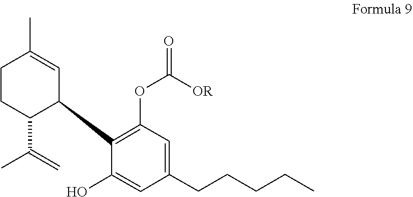
wherein R is the therapeutic counterpart.

22. The cannabinoid compound of claim 2, wherein the cannabinoid compound is a compound of formula 10: 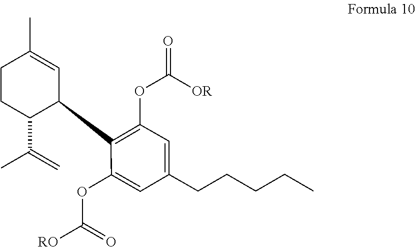
wherein each R is the therapeutic counterpart, selected independently.

23. The cannabinoid compound of claim 2, wherein the cannabinoid compound is a compound of formula 11: 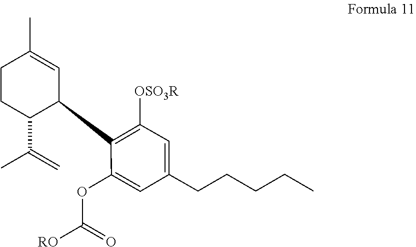
wherein each R is the therapeutic counterpart, selected independently.

24. The cannabinoid compound of claim 2, wherein the cannabinoid compound is a compound of formula 12: 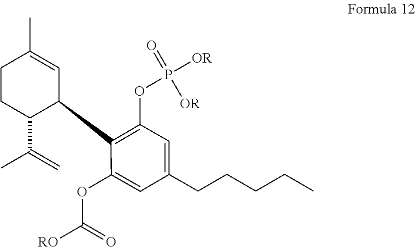
wherein each R is the therapeutic counterpart, selected independently.

25. The cannabinoid compound of claim 2, wherein the cannabinoid compound is a compound of formula 13: 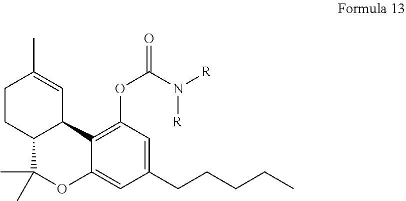
wherein each R is the therapeutic counterpart, selected independently.

26. The cannabinoid compound of claim 2, wherein the cannabinoid compound is a compound of formula 14: 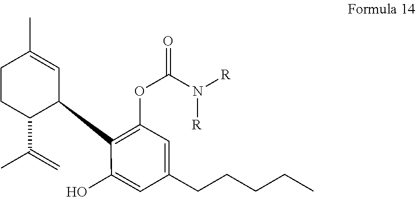
wherein each R is the therapeutic counterpart, selected independently.

27. The cannabinoid compound of claim 2, wherein the cannabinoid compound is a compound of formula 15: 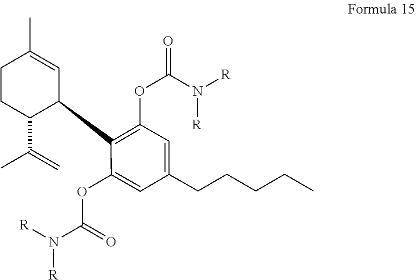
wherein each R is the therapeutic counterpart, selected independently.

28. The cannabinoid compound of claim 2, wherein the cannabinoid compound is a compound of formula 16: 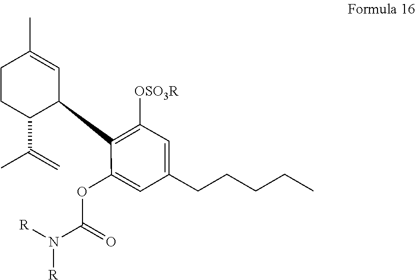
wherein each R is the therapeutic counterpart, selected independently.

29. The cannabinoid compound of claim 2, wherein the cannabinoid compound is a compound of formula 17: 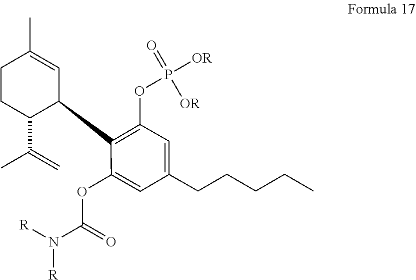
wherein each R is the therapeutic counterpart, selected independently.

30. The cannabinoid compound of claim 2, wherein the cannabinoid compound is a compound of formula 18:

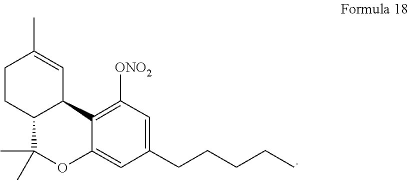

31. The cannabinoid compound of claim 2, wherein the cannabinoid compound is a compound of formula 19:

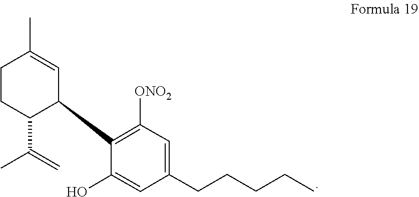

32. The cannabinoid compound of claim 2, wherein the cannabinoid compound is a compound of formula 20:

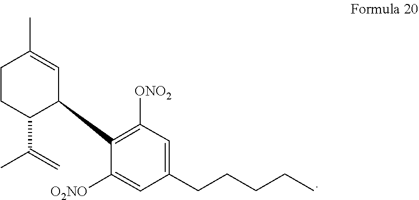

33. The cannabinoid compound of claim 2, wherein the cannabinoid compound is a compound of formula 21: 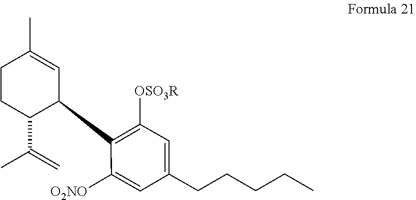
wherein R is the therapeutic counterpart.

34. The cannabinoid compound of claim 2, wherein the cannabinoid compound is a compound of formula 22: 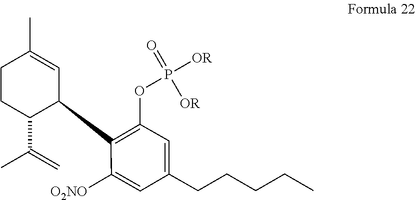
wherein each R is the therapeutic counterpart, selected independently.

35. The cannabinoid compound of claim 2, wherein the cannabinoid compound is a compound of formula 23: 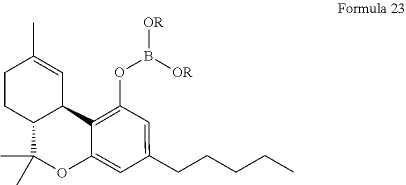
wherein each R is the therapeutic counterpart, selected independently.

36. The cannabinoid compound of claim 2, wherein the cannabinoid compound is a compound of formula 24: 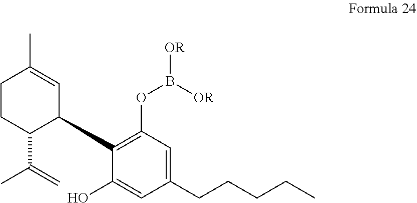
wherein each R is the therapeutic counterpart, selected independently.

37. The cannabinoid compound of claim 2, wherein the cannabinoid compound is a compound of formula 25: 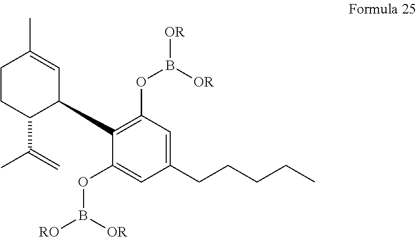
wherein each R is the therapeutic counterpart, selected independently.

38. The cannabinoid compound of claim 2, wherein the cannabinoid compound is a compound of formula 26: 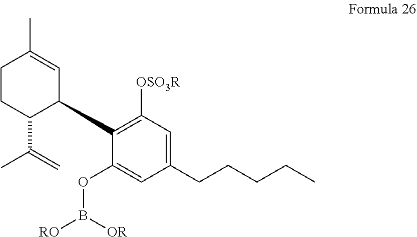
wherein each R is the therapeutic counterpart, selected independently.

39. The cannabinoid compound of claim 2, wherein the cannabinoid compound is a compound of formula 27: 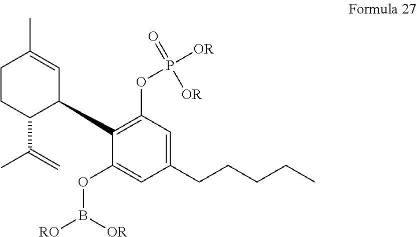
wherein each R is the therapeutic counterpart, selected independently.

40. The cannabinoid compound of claim 2, wherein the cannabinoid compound is a compound of formula 28: 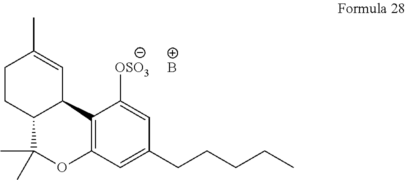
wherein B is the therapeutic counterpart.

41. The cannabinoid of claim 40, wherein the therapeutic counterpart is selected from the group consisting of: glucosamine, psilocin, pregabalin, gabapentin, topiramate, morphine, levodopa, and citalopram.

42. The cannabinoid compound of claim 2, wherein the cannabinoid compound is a compound of formula 29: 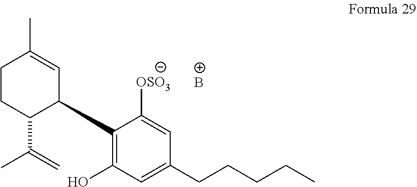
wherein B is the therapeutic counterpart.

43. The cannabinoid of claim 42, wherein the therapeutic counterpart is selected from the group consisting of: glucosamine, psilocin, pregabalin, gabapentin, topiramate, morphine, levodopa, and citalopram.

44. The cannabinoid compound of claim 2, wherein the cannabinoid compound is a compound of formula 30: 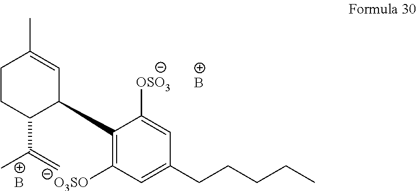
wherein each B is the therapeutic counterpart, selected independently.

45. The cannabinoid compound of claim 2, wherein the cannabinoid compound is a compound of formula 31: 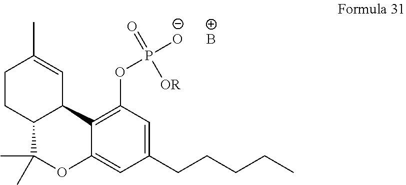
wherein each R and B is the therapeutic counterpart, selected independently.

46. The cannabinoid compound of claim 2, wherein the cannabinoid compound is a compound of formula 32: 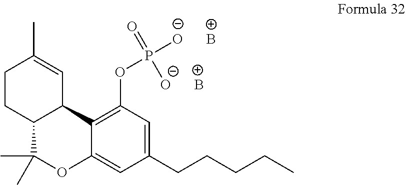
wherein each B is the therapeutic counterpart, selected independently.

47. The cannabinoid of claim 46, wherein the therapeutic counterpart is selected from the group consisting of: glucosamine, psilocin, pregabalin, gabapentin, topiramate, morphine, levodopa, and citalopram.

48. The cannabinoid compound of claim 2, wherein the cannabinoid compound is a compound of formula 33: 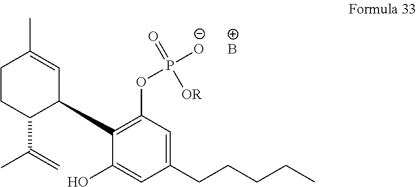
wherein each R and B is the therapeutic counterpart, selected independently.

49. The cannabinoid compound of claim 2, wherein the cannabinoid compound is a compound of formula 34: 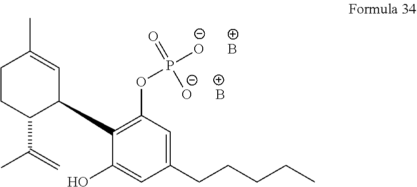
wherein each B is the therapeutic counterpart, selected independently.

50. The cannabinoid of claim 49, wherein the therapeutic counterpart is selected from the group consisting of: glucosamine, psilocin, pregabalin, gabapentin, topiramate, morphine, levodopa, and citalopram.

51. The cannabinoid compound of claim 2, wherein the cannabinoid compound is a compound of formula 35: 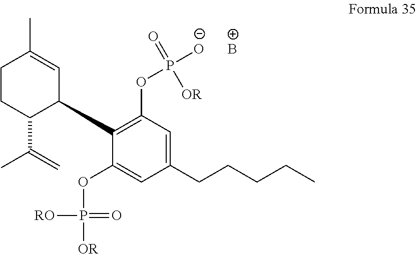
wherein each R and B is the therapeutic counterpart, selected independently.

52. The cannabinoid compound of claim 2, wherein the cannabinoid compound is a compound of formula 36: 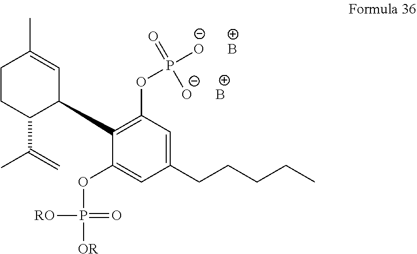
wherein each R and B is the therapeutic counterpart, selected independently.

53. The cannabinoid compound of claim 2, wherein the cannabinoid compound is a compound of formula 37: 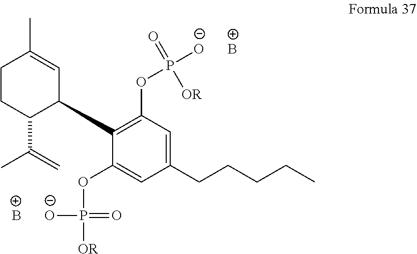
wherein each R and B is the therapeutic counterpart, selected independently.

54. The cannabinoid compound of claim 2, wherein the cannabinoid compound is a compound of formula 38: 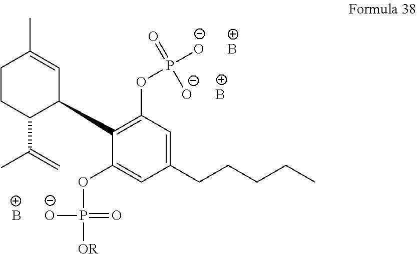
wherein each R and B is the therapeutic counterpart, selected independently.

55. The cannabinoid compound of claim 2, wherein the cannabinoid compound is a compound of formula 39: 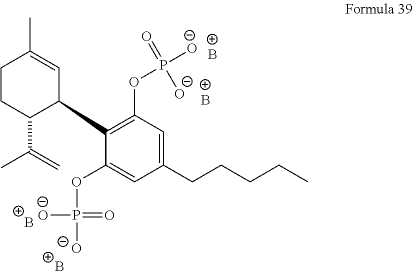
wherein each B is the therapeutic counterpart, selected independently.

56. The cannabinoid compound of claim 2, wherein the cannabinoid compound is a compound of formula 40: 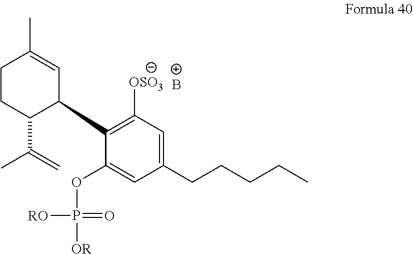
wherein each R and B is the therapeutic counterpart, selected independently.

57. The cannabinoid compound of claim 2, wherein the cannabinoid compound is a compound of formula 41: 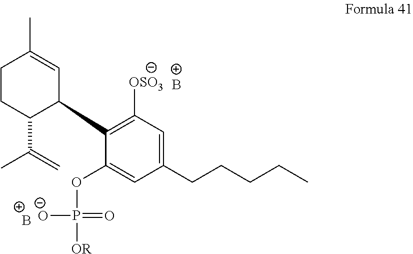
wherein each R and B is the therapeutic counterpart, selected independently.

58. The cannabinoid compound of claim 2, wherein the cannabinoid compound is a compound of formula 42: 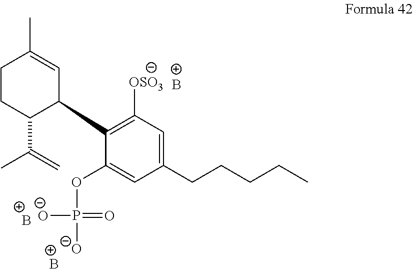
wherein each B is the therapeutic counterpart, selected independently.

59. The cannabinoid compound of claim 2, wherein the cannabinoid compound is a compound of formula 43: 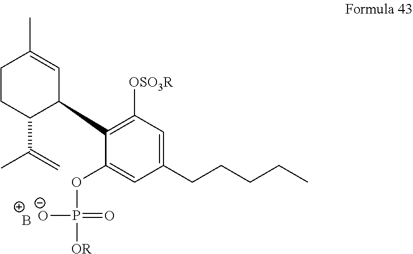
wherein each R and B is the therapeutic counterpart, selected independently.

60. The cannabinoid compound of claim 2, wherein the cannabinoid compound is a compound of formula 44: 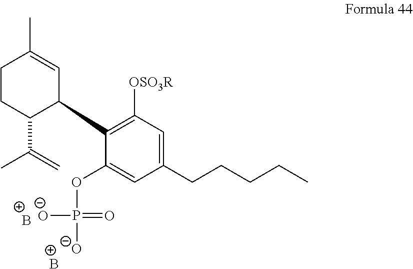
wherein each R and B is the therapeutic counterpart, selected independently.

61. The cannabinoid compound of claim 2, wherein the cannabinoid compound is a compound of formula 45: 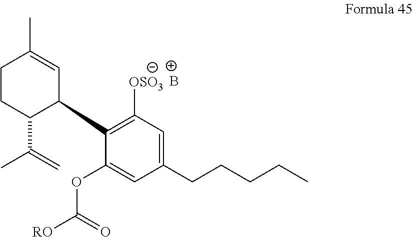
wherein each R and B is the therapeutic counterpart, selected independently.

62. The cannabinoid compound of claim 2, wherein the cannabinoid compound is a compound of formula 46: 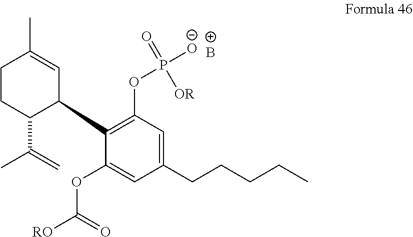
wherein each R and B is the therapeutic counterpart, selected independently.

63. The cannabinoid compound of claim 2, wherein the cannabinoid compound is a compound of formula 47: 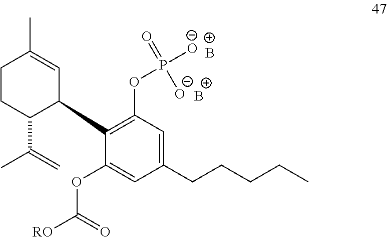
wherein each R and B is the therapeutic counterpart, selected independently.

64. The cannabinoid compound of claim 2, wherein the cannabinoid compound is a compound of formula 48: 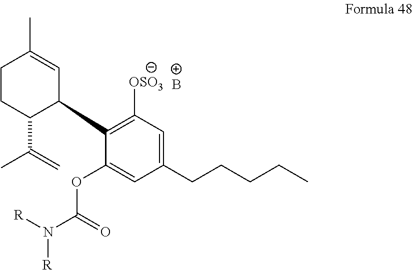
wherein each R and B is the therapeutic counterpart, selected independently.

65. The cannabinoid compound of claim 2, wherein the cannabinoid compound is a compound of formula 49: 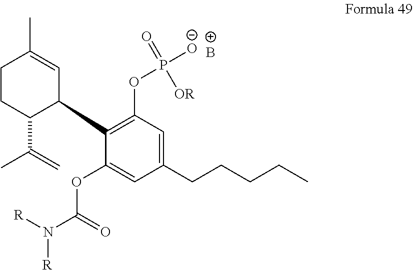
wherein each R and B is the therapeutic counterpart, selected independently.

66. The cannabinoid compound of claim 2, wherein the cannabinoid compound is a compound of formula 50: 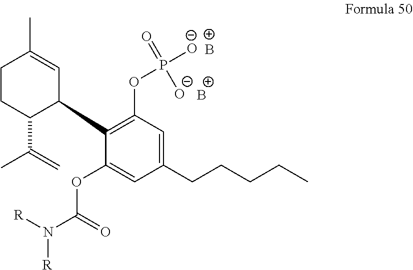
wherein each R and B is the therapeutic counterpart, selected independently.

67. The cannabinoid compound of claim 2, wherein the cannabinoid compound is a compound of formula 51: 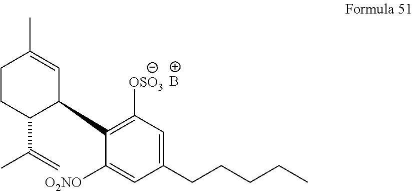
wherein B is the therapeutic counterpart.

68. The cannabinoid compound of claim 2, wherein the cannabinoid compound is a compound of formula 52: 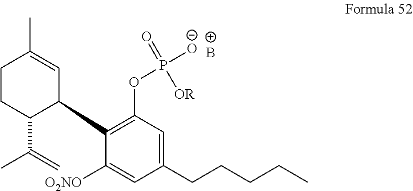
wherein each R and B is the therapeutic counterpart, selected independently.

69. The cannabinoid compound of claim 2, wherein the cannabinoid compound is a compound of formula 53: 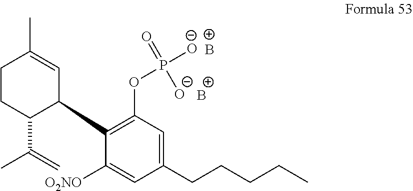
wherein each B is the therapeutic counterpart, selected independently.

70. The cannabinoid compound of claim 2, wherein the cannabinoid compound is a compound of formula 54: 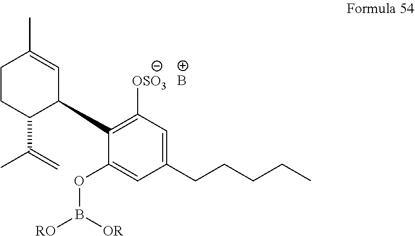
wherein each R and counter ion B is the therapeutic counterpart, selected independently.

71. The cannabinoid compound of claim 2, wherein the cannabinoid compound is a compound of formula 55: 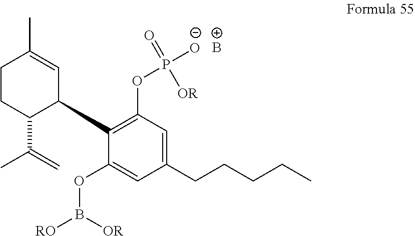
wherein each R and counter ion B is the therapeutic counterpart, selected independently.

72. The cannabinoid compound of claim 2, wherein the cannabinoid compound is a compound of formula 56: 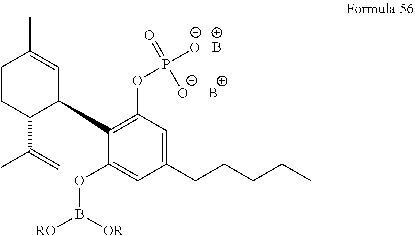
wherein each R and counter ion B is the therapeutic counterpart, selected independently.

73. The cannabinoid compound of claim 2, wherein the cannabinoid compound is a compound of formula 57: 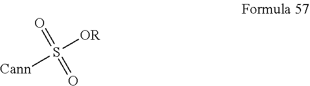
wherein Cann is the cannabinoid and R is the therapeutic counterpart.

74. The cannabinoid compound of claim 73, wherein the cannabinoid compound is a compound of formula 58: 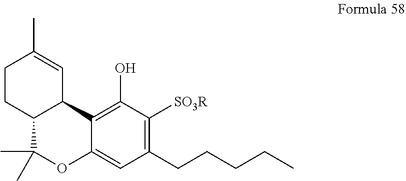
wherein R is the therapeutic counterpart.

75. The cannabinoid compound of claim 73, wherein the cannabinoid compound is a compound of formula 59: 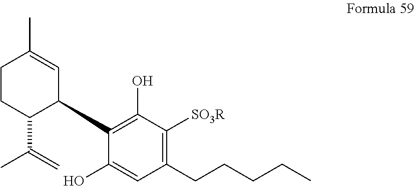
wherein R is the therapeutic counterpart.

76. The cannabinoid compound of claim 2, wherein the cannabinoid compound is a compound of formula 60: 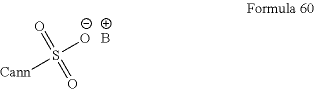
wherein Cann is the cannabinoid and B is the therapeutic counterpart.

77. The cannabinoid compound of claim 76, wherein the cannabinoid compound is a compound of formula 61: 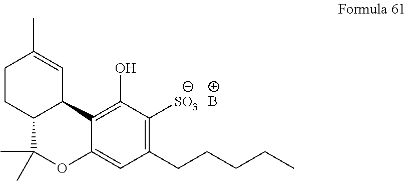
wherein B is the therapeutic counterpart. 10 78. The cannabinoid of claim 77, wherein the therapeutic counterpart is selected from the group consisting of: glucosamine, psilocin, pregabalin, gabapentin, topiramate, morphine, levodopa, and citalopram.

79. The cannabinoid compound of claim 76, wherein the cannabinoid compound is a compound of formula 62: 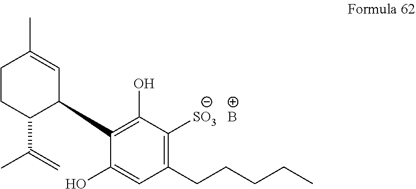
wherein B is the therapeutic counterpart.

80. The cannabinoid of claim 79, wherein the therapeutic counterpart is selected from the group consisting of: glucosamine, psilocin, pregabalin, gabapentin, topiramate, morphine, levodopa, and citalopram.

81. The cannabinoid compound of claim 2, wherein the cannabinoid compound is a compound of formula 63: 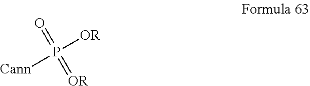
wherein each R is the therapeutic counterpart, selected independently.

82. The cannabinoid compound of claim 81, wherein the cannabinoid compound is a compound of formula 64: 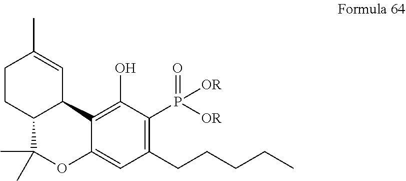
wherein each R is the therapeutic counterpart, selected independently.

83. The cannabinoid compound of claim 81, wherein the cannabinoid compound is a compound of formula 65: 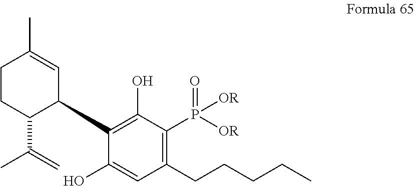
wherein each R is the therapeutic counterpart, selected independently.

84. The cannabinoid compound of claim 2, wherein the cannabinoid compound is a compound of formula 66: 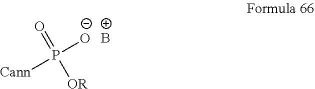
wherein Cann is the cannabinoid and each R and B is the therapeutic counterpart, selected independently.

85. The cannabinoid compound of claim 84, wherein the cannabinoid compound is a compound of formula 68: 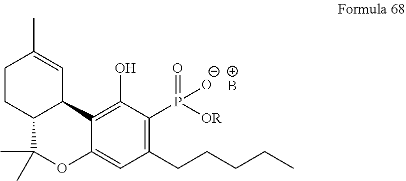
wherein each R and B is the therapeutic counterpart, selected independently.

86. The cannabinoid compound of claim 84, wherein the cannabinoid compound is a compound of formula 70: 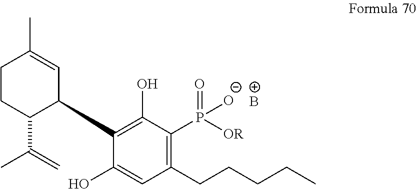
wherein each R and B is the therapeutic counterpart, selected independently.

87. The cannabinoid compound of claim 2, wherein the cannabinoid compound is a compound of formula 67: 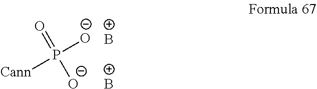
wherein each B is the therapeutic counterpart, selected independently.

88. The cannabinoid compound of claim 87, wherein the cannabinoid compound is a compound of formula 69: 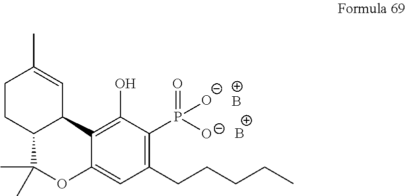
wherein each B is the therapeutic counterpart, selected independently.

89. The cannabinoid of claim 88, wherein the therapeutic counterpart is selected from the group consisting of: glucosamine, psilocin, pregabalin, gabapentin, topiramate, morphine, levodopa, and citalopram.

90. The cannabinoid compound of claim 87, wherein the cannabinoid compound is a compound of formula 71: 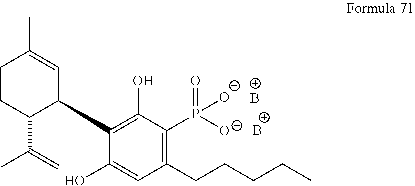
wherein each B is the therapeutic counterpart, selected independently.

91. The cannabinoid of claim 90, wherein the therapeutic counterpart is selected from the group consisting of: glucosamine, psilocin, pregabalin, gabapentin, topiramate, morphine, levodopa, and citalopram.

92. The cannabinoid compound of claim 2, wherein the cannabinoid compound is a compound of formula 72: 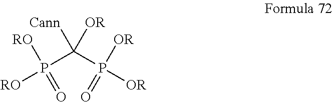
wherein Cann is the cannabinoid and each R is the therapeutic counterpart, selected independently.

93. The cannabinoid compound of claim 2, wherein the cannabinoid compound is a compound of formula 73: 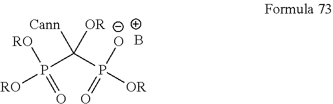
wherein Cann is the cannabinoid and each R and B is the therapeutic counterpart, selected independently.

94. The cannabinoid compound of claim 2, wherein the cannabinoid compound is a compound of formula 74: 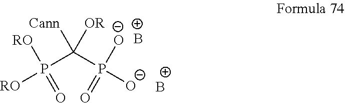
wherein Cann is the cannabinoid and each R and B is the therapeutic counterpart, selected independently.

95. The cannabinoid compound of claim 2, wherein the cannabinoid compound is a compound of formula 75: 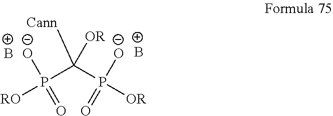
wherein Cann is the cannabinoid and each R and B is the therapeutic counterpart, selected independently.

96. The cannabinoid compound of claim 2, wherein the cannabinoid compound is a compound of formula 76: 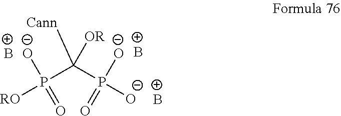
wherein Cann is the cannabinoid and each R and B is the therapeutic counterpart, selected independently.

97. The cannabinoid compound of claim 2, wherein the cannabinoid compound is a compound of formula 77: 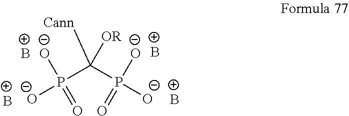
wherein Cann is the cannabinoid and each B is the therapeutic counterpart, selected independently.

98. The cannabinoid compound of claim 2, wherein the cannabinoid compound is a compound of formula 78: 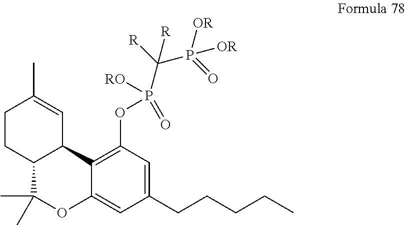
wherein each R is the therapeutic counterpart, selected independently.

99. The cannabinoid compound of claim 2, wherein the cannabinoid compound is a compound of formula 79: 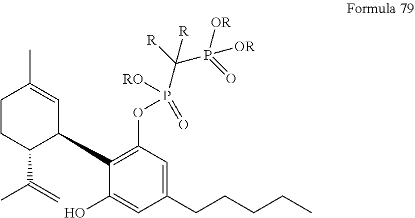
wherein each R is the therapeutic counterpart, selected independently.

100. The cannabinoid compound of claim 2, wherein the cannabinoid compound is a compound of formula 80: 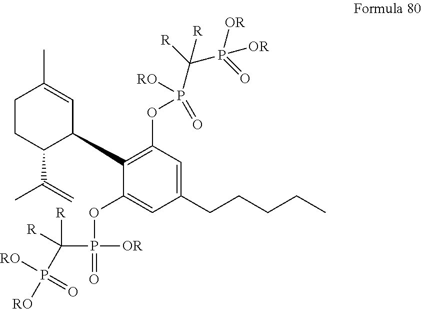
wherein each R is the therapeutic counterpart, selected independently.

101. The cannabinoid compound of claim 2, wherein the cannabinoid compound is a compound of formula 81: 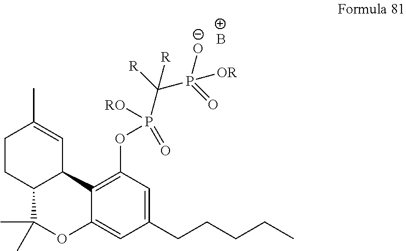
wherein each R and B is the therapeutic counterpart, selected independently.

102. The cannabinoid compound of claim 2, wherein the cannabinoid compound is a compound of formula 82: 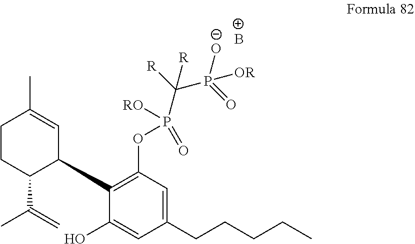
wherein each R and B is the therapeutic counterpart, selected independently.

103. The cannabinoid compound of claim 2, wherein the cannabinoid compound is a compound of formula 83: 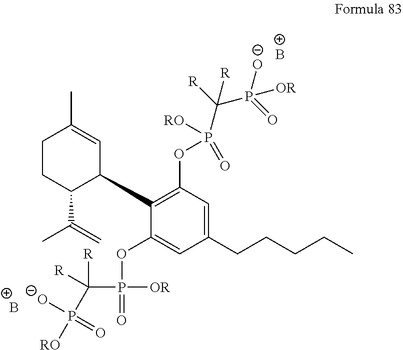
wherein each R and B is the therapeutic counterpart, selected independently.

104. A pharmaceutical formulation comprising a labile ester of a cannabinoid with a first synergistic or additive therapeutic counterpart and a second active ingredient having a synergistic or additive effect with the cannabinoid.

105. The pharmaceutical formulation of claim 104, wherein the second active ingredient is selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), Boswellia sp., Boswellia carterii, Boswellia serrata, ginger, capsaicin, camphor, polyphenols, quercetin, ellagic acid, curcumin, and resveratrol, phytosterols, carbohydrates, including mannose-6-phosphate, essential oils, including thymol, and carvacrol, terpenoids, including squalene, lycopene, p-cymene, linalool, and derivatives, analogues, salts, mixtures, and combinations thereof.

106. The pharmaceutical formulation of claim 104, wherein the pharmaceutical formulation is in the form of a hard gelatin capsule, comprising 25 mg of glucosamine CBD-sulfate, 750 mg of glucosamine sulfate, 315 mg of microcrystalline cellulose pH102, 30 mg of magnesium stearate, and 30 mg of silicon dioxide.

107. The pharmaceutical formulation of claim 104, wherein the pharmaceutical formulation is in the form of a hard gelatin capsule, comprising 25 mg of glucosamine CBD-sulfate, 750 mg of glucosamine sulfate, 600 mg of chondroitin sulfate, 300 mg of methylsulfonylmethane, 140 mg of microcrystalline cellulose pH102, 30 mg of magnesium stearate, and 30 mg of silicon dioxide.

108. The pharmaceutical formulation of claim 104, wherein the pharmaceutical formulation is in the form of a hard gelatin capsule, comprising 25 mg of glucosamine CBD-sulfate, 750 mg of glucosamine sulfate, 600 mg of chondroitin sulfate, 140 mg of microcrystalline cellulose pH102, 60 mg of providone K30, 60 mg of croscarmellose sodium, 30 mg of magnesium stearate, and 30 mg of silicon dioxide.

109. The pharmaceutical formulation of claim 104, wherein the pharmaceutical formulation is in the form of a hard gelatin capsule, comprising 25 mg of glucosamine CBD-sulfate, 750 mg of glucosamine sulfate, 600 mg of chondroitin sulfate, 300 mg of methylsulfonylmethane, 200 mg of collagen, 140 mg of microcrystalline cellulose pH102, 30 mg of magnesium stearate, and 30 mg of silicon dioxide.

110. The pharmaceutical formulation of claim 104, wherein the pharmaceutical formulation is in the form of a tablet, comprising 25 mg of glucosamine CBD-sulfate, 120 mg of pregelatinized starch, 300 mg of mannitol, 20 mg of copovidone, 5 mg of talc, and 5 mg of silicon dioxide.

111. The pharmaceutical formulation of claim 104, wherein the pharmaceutical formulation is in the form of a cream, comprising 750 mg of histamine CBD-sulfate, 2500 mg of glucosamine sulfate, 0.75 mg of sorbitan monostearate, 3 mg of tween 60, 6 mg of cetostearyl alcohol, 5 mg of propylene glycol, 1 mg of benzyl alcohol, 0.14 mg of methyl paraben, 0.02 mg of butylated hydroxytoluene, 8 mg of medium chain triglycerides, 5 mg of isopropyl myristate, and purified water to fill per 100 g of the cream.

112. A method of producing a cannabinoid compound, the cannabinoid compound comprising a labile ester of a cannabinoid with a synergistic or additive therapeutic counterpart, comprising the step of: mixing a cannabinoid ester salt having a labile counter ion with the therapeutic counterpart in the form of a base, wherein the mixing step takes place in the presence of an aqueous solvent.

113. The method of claim 112, wherein the aqueous solvent is a 1:1 mixture of aqueous and non-aqueous solvent.

114. The method of claim 112, wherein the therapeutic counterpart is added in the mixing step in an amount greater than the amount of the cannabinoid ester salt.

115. The method of claim 114, wherein the ratio of the cannabinoid ester salt to the therapeutic counterpart added in the mixing step is 1:1.2.

116. The method of claim 112, wherein the cannabinoid ester salt is a cannabinoid sulfate ester salt, and wherein the labile counter ion is pyridine, and the method further comprises an earlier step of producing the cannabinoid sulfate ester salt by mixing a cannabinoid with pyridine sulfur trioxide in pyridine.

117. The method of claim 116, wherein the earlier step of producing the cannabinoid sulfate ester salt takes place at an elevated temperature and pressure above room temperature and atmospheric pressure.

118. The method of claim 117, wherein the elevated temperature and pressure is between 65-90° C. and between 5-20 bar.

119. The method of claim 118, wherein the cannabinoid is THC and the therapeutic counterpart is glucosamine.

120. The method of claim 118, wherein the cannabinoid is CBD and the therapeutic counterpart is glucosamine.

121. The method of claim 118, wherein the cannabinoid is THC and the therapeutic counterpart is psilocin.

122. The method of claim 118, wherein the cannabinoid is CBD and the therapeutic counterpart is psilocin.

123. The method of claim 118, wherein the cannabinoid is THC and the therapeutic counterpart is gabapentin or a related gabapentinoid.

124. The method of claim 118, wherein the cannabinoid is CBD and the therapeutic counterpart is gabapentin or a related gabapentinoid.

125. The method of claim 118, wherein the cannabinoid is THC and the therapeutic counterpart is pregabalin or a related gabapentinoid.

126. The method of claim 118, wherein the cannabinoid is CBD and the therapeutic counterpart is pregabalin or a related gabapentinoid.

127. The use of a cannabinoid compound of claims 1-103 or a pharmaceutical formulation of claims 104-111 to treat, alleviate, or reduce the symptoms of a human or animal subject suffering from one or more conditions, disorders, or illnesses selected from the group consisting of: pain, neuropathic pain, inflammation, neurodegenerative disorders, multiple sclerosis, spinal cord and brain injury, post-traumatic stress disorder, epilepsy, paediatric seizure disorders, addiction, insomnia, nausea and vomiting, cancer, renal fibrosis, obesity, schizophrenia, depression, obsessive compulsive disorders, anxiety, psychiatric disorders, sleep disorders, fibromyalgia, Tourette syndrome, glaucoma, Crohn's disease, inflammatory bowel disorders, cluster headache, and anorexia.
